(19) United States
(12) Patent Application Publication
(10) Pub. No.: US 2012/0095193 A1
(43) Pub. Date: Apr. 19, 2012

(54) POLYPEPTIDES AND METHOD OF TREATMENT

(76) Inventors: Michael Neil Burden, Hertfordshire (GB); Paul Andrew Hamblin, Hertfordshire (GB); Jonathan David Larkin, King of Prussia, PA (US); John Richard White, King of Prussia, PA (US)

(21) Appl. No.: 13/379,748

(22) PCT Filed: Jul. 1, 2010
(86) PCT No.: PCT/US10/40724

§ 371 Date: Dec. 21, 2011

Related U.S. Application Data
(60) Provisional application No. 61/222,521, filed on Jul. 2, 2009.
(60) Provisional application No. 61/249,757, filed on Oct. 8, 2009.

(51) Int. Cl. C07K 16/40; C12N 1/15; C12N 1/21; C12N 1/19; C12N 15/13; C12N 5/10

(52) U.S. Cl. 530/387.3; 530/387.9; 536/23.53; 435/331; 435/252.3; 435/252.31; 435/252.35; 435/254.2; 435/254.11

(57) ABSTRACT

Provided herein are isolated antigen binding protein comprising at least one first immunoglobulin variable domain capable of binding to human ADAMTS5. Also provided are antigen binding proteins of the present invention that are monoclonal antibodies, pharmaceutical compositions comprising said antigen binding proteins and methods of treatment.

### FIELD OF THE INVENTION

This invention relates to methods for inhibiting aggrecanase, in particular ADAMTS5, thereby reducing the break down of aggrecan in cartilage.

### BACKGROUND OF THE INVENTION

Cartilage is an avascular tissue populated by specialized cells termed chondrocytes, which respond to diverse mechanical and biochemical stimuli. Cartilage is present in the linings of joints, interstitial connective tissues, and basement membranes, and is composed of an extracellular matrix comprised of several matrix components including type II collagen, proteoglycans, fibronectin and laminin.

In normal cartilage, extracellular matrix synthesis is offset by extracellular matrix degradation, resulting in normal matrix turnover. Depending on the signal(s) received, the ensuing response may be either anabolic (leading to matrix production and/or repair) or catabolic (leading to matrix degradation, cellular apoptosis, loss of function, and pain).

In response to injurious compression and/or exposure to inflammatory mediators (e.g. inflammatory cytokines) chondrocytes decrease matrix production and increase production of multiple matrix degrading enzymes. Examples of matrix degrading enzymes include aggrecanases (ADAMTSs) and matrix metalloproteases (MMPs). The activities of these enzymes result in the degradation of the cartilage matrix. Aggrecanases (ADAMTSs), in conjunction with MMPs, degrade aggrecan, an aggregating proteoglycan present in articular cartilage. In osteoarthritic (OA) articular cartilage a loss of proteoglycan staining is observed in the superficial zone in early OA and adjacent to areas of cartilage erosion in moderate to severe OA.

Aggrecan catabolism as mediated by aggrecanase occurs at certain conserved sites in aggrecan. Human ADAMTS4 (shown in FIG. 5 as SEQ ID NO:44) and ADAMTS5 (shown in FIG. 4 as SEQ ID NO:43) have been shown to cleave aggrecan between amino acids E373 and A374 producing the neoepitope ARGSVIL (SEQ ID NO:1).

Excessive degradation of extracellular matrix is implicated in the pathogenesis of many diseases and conditions, including pain, chronic pain, neuropathic pain, postoperative pain, rheumatoid arthritis, osteoarthritis, sports injuries, erosive arthritis, ankylosing spondylosis, neuralgia, neuropathies, algesia, nerve injury, ischaemia, neurodegeneration, cartilage degeneration, stroke, incontinence, inflammatory disorders, irritable bowel syndrome, periodontal disease, aberrant angiogenesis, tumor invasion and metastasis, corneal ulceration, and in complications of diabetes.

Thus, there is a need for compounds capable of inhibiting aggrecanase activity and cartilage degradation.

### SUMMARY OF THE INVENTION

The present invention provides isolated polypeptides comprising at least one variable domain capable of binding and/or neutralizing human ADAMTS5.

Also provided are isolated polynucleotides encoding the polypeptides of the present invention.

Also, provided are pharmaceutical compositions comprising at least one polypeptide of the present invention.

Methods are provided herein for treating a patient suffering from a disease of the cartilage with a pharmaceutical composition of the present invention.

### BRIEF DESCRIPTION OF THE DRAWINGS

FIG. 1: In vitro Inhibition of ARGSVIL (SEQ ID NO:1) Neoepitope Generation by ADAMTS5 mAbs.

FIG. 2: In vitro Concentration Dependent Inhibition of ARGSVIL (SEQ ID NO:1) Neoepitope Generation by 7B4.1E11 Murine mAb

FIG. 3: Mean Total Joint Score for Mice Treated with Selected ADAMTS5 Antibodies versus Control In vivo

FIG. 4: Amino Acid sequence of human ADAMTS5 (SEQ ID NO:43).

FIG. 5: Amino Acid sequence of Human ADAMTS4 (SEQ ID NO:44).

FIG. 6: Binding of the humanized anti ADAMTS5 antibodies to recombinant antigen.

FIG. 7: Binding of the humanized anti ADAMTS5 antibodies to recombinant antigen.

FIG. 8: Binding of the humanized anti ADAMTS5 antibodies to recombinant antigen.

FIG. 9: Binding of the humanized anti ADAMTS5 antibodies to recombinant antigen.

FIG. 10: Percent Inhibition of ADAMTS5 activity.

FIG. 11: Percent Inhibition of ADAMTS5 activity.

FIG. 12: Binding of the purified anti ADAMTS5 antibodies to recombinant antigen.

FIG. 13: Structure modeling predicts Ag/Ab interaction sites.

### DETAILED DESCRIPTION OF THE INVENTION

“Polynucleotide” generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids. “Polypeptides” include amino acid sequences modified either by natural processes, such as posttranslational processing, or by chemical modification techniques that are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS-STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter, et al., “Analysis for protein modifications and nonprotein cofactors”, Meth. Enzymol. (1990) 182:626-646 and Rattan, et al., “Protein Synthesis: Posttranslational Modifications and Aging”, Ann NY Acad Sci (1992) 663:48-62.

“Variant” as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” including, but not limited to, when such polynucleotide or polypeptide is introduced back into a cell.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered, for example, increased, decreased or eliminated. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

As used herein “inflammatory mediators” include any compound capable of triggering an inflammatory process. The term inflammation generally refers to the process of reaction of vascularized living tissue to injury. This process includes but is not limited to increased blood flow, increased vascular permeability, and leukocytic exudation. Because leukocytes recruited into inflammatory reactions can release potent enzymes and oxygen free radicals (i.e. inflammatory mediators), the inflammatory response is capable of mediating considerable tissue damage. Examples of inflammatory mediators include, but are not limited to prostaglandins (e.g. PGE2), leukotrienes (e.g. LTB4), inflammatory cytokines, such as tumour necrosis factor alpha (TNFα), interleukin 1 (IL-1), and interleukin 6 (IL-6); nitric oxide (NO), metalloproteinases, and heat shock proteins.

As used herein “matrix protein” includes proteins released from cells to form the extracellular matrix of cartilage. The extracellular matrix of cartilage consists of proteoglycans, belonging to several distinct proteoglycan families. These include, but are not limited to, perlecan and the hyalectans, exemplified by aggrecan and versican, and the small leucine-rich family of proteoglycans, including decorin, biglycan and fibromodulin. The extracellular matrix also consists of hybrid collagen fibers comprised of three collagen isotypes, namely type II, type IX, and type XI collagens, along with accessory proteins such as cartilage oligeromeric matrix protein (COMP), link protein, and fibronectin. Cartilage also contains hyaluronin which forms a noncovalent association with the hyalectins. In addition, a specialized pericellular matrix surrounds the chondrocyte which consists of proteoglycans, type VI collagen and collagen receptor proteins, such as anchorin.

As used herein “matrix degrading enzymes” refers to enzymes able to cleave extracellular matrix proteins. Cartilage extracellular matrix turnover is regulated by matrix metalloproteases (MMPs) which are synthesized as latent proenzymes that require activation in order to degrade cartilage extracellular matrix proteins. Three classes of enzymes are believed to regulate the turnover of extracellular matrix proteins, namely collagenases (including, but not limited to, MMP-13), responsible for the degradation of native collagen fibers, stromelysins (including, but not limited to, MMP-3) which degrade proteoglycan and type IX collagen, and gelatinases (including, but not limited to, MMP-2 and MMP-9) which degrade denatured collagen. The matrix degrading enzyme group that appears most relevant in cartilage degradation in OA includes a subgroup of metalloproteinases called ADAMTS, because they possess disintegrin and metalloproteinase domains and a thrombospondin motif in their structure. ADAMTS4 (aggrecanase-1) has been reported to be elevated in OA joints and along with ADAMTS-5 (aggrecanase-2) have been shown to be expressed in human osteoarthritic cartilage. These enzymes appear to be responsible for aggrecan degradation without MMP participation.

As used herein, “reduce” or “reducing” aggrecanase activity refers to a decrease in any and/or all of the activities associated with at least one naturally occurring aggrecanase, including but not limited to ADAMTS4 and ADAMTS5. For example “reducing” at least one ADAMTS5 activity refers to a decrease in any and/or all of the activities associated with naturally occurring ADAMTS5. By way of example, reducing ADAMTS5 in a mammal activity can be measured after administration of at least one polypeptide capable of binding to ADAMTS5 to a subject and compared with ADAMTS5 activity in the same subject prior to the administration of the polypeptide capable of binding to ADAMTS5 or in comparison to a second subject who is administered placebo. As used herein, “reducing” at least one ADAMTS5 includes the reduction of at least one or more enzyme activity. A reduction in at least one ADAMTS5 activity includes a complete abrogation of at least one ADAMTS5. Also included within this definition is a reduced amount of at least one enzyme activity. That is, ADAMTS5 may have more than one activity which is maintained the while a second activity of the same enzyme is reduced.

As used herein “diseases associated with cartilage degradation” include, but are not limited to cancer, pain, chronic pain, neuropathic pain, postoperative pain, osteoarthritis, sports injuries, erosive arthritis, rheumatoid arthritis, psoriatic arthritis, Lyme arthritis, juvenile arthritis, ankylosing spondylosis, neuralgia, neuropathies, algesia, nerve injury, ischaemia, neurodegeneration, inflammatory diseases, cartilage degeneration, diseases affecting the larynx, trachea, auditory canal, intervertebral discs, ligaments, tendons, joint capsules or bone development, invertebral disc degeneration, osteopenia, or periodontal diseases, acute joint injury, and/or a disease related to joint destruction.

As used herein “co-administration” or “co-administering” as used herein refers to administration of two or more compounds to the same patient. Co-administration of such compounds may be simultaneous or at about the same time (e.g., within the same hour) or it may be within several hours or days of one another. For example, a first compound may be administered once weekly while a second compound is co-administered daily.

As used herein “attenuate” or “attenuating” refers to a normalization (i.e., either an increase or decrease) of the amount of matrix degrading enzyme, inflammatory mediator, or matrix protein produced and/or released by a cell, following exposure to a catabolic stimulus. For example, following exposure to IL-1 chondrocyte production of matrix proteins, such as proteoglycans, are reduced, while production of matrix degrading enzymes (e.g. MMP-13, ADAMTS4) and reactive oxygen species (e.g. NO) are increased. Attenuation refers to the normalization of these diverse responses to levels observed in the absence of a catabolic stimulus.

A “domain antibody” or “dAb” may be considered the same as a “single variable domain” which is capable of binding to an antigen. A single variable domain may be a human antibody variable domain, but also includes single antibody variable domains from other species such as rodent (for example, as disclosed in WO 00/29004), nurse shark and Camelid VHH dAbs. Camelid VHH are immunoglobulin single variable domain polypeptides that are derived from species including camel, llama, alpaca, dromedary, and guanaco, which produce heavy chain antibodies naturally devoid of light chains. Such VHH domains may be humanized according to standard techniques available in the art, and such domains are considered to be “domain antibodies.” As used herein VH includes camelid VHH domains.

The phrase “single variable domain” refers to an antigen binding protein variable domain (for example, V_{H}, V_{HH}, V_{L}) that specifically binds an antigen or epitope independently of a different variable region or domain.

The term “antigen binding protein” as used herein refers to antibodies, antibody fragments and other protein constructs, such as domains, but not limited to, variable domains and domain antibodies, which are capable of binding to an antigen.

The antigen binding domain of an antibody comprises two separate regions: a heavy chain variable domain (V_{H}) and a light chain variable domain (V_{L}: which can be either V_{κ} or V_{λ}). The antigen binding site itself is formed by six polypeptide loops: three from V_{H} domain (H1, H2 and H3) and three from V_{L} domain (L1, L2 and L3).

Analysis of the structures and sequences of antibodies has shown that five of the six antigen binding loops (H1, H2, L1, L2, L3) possess a limited number of main-chain conformations or canonical structures (Chothia and Lesk (1987) J. Mol. Biol., 196: 901; Chothia et al. (1989) Nature, 342: 877). The main-chain conformations are determined by (i) the length of the antigen binding loop, and (ii) particular residues, or types of residue, at certain key position in the antigen binding loop and the antibody framework. Analysis of the loop lengths and key residues has enabled us to the predict the main-chain conformations of H1, H2, L1, L2 and L3 encoded by the majority of human antibody sequences (Chothia et al. (1992) J. Mol. Biol., 227: 799; Tomlinson et al. (1995) EMBO J., 14: 4628; Williams et al. (1996) J. Mol. Biol., 264: 220). Although the H3 region is much more diverse in terms of sequence, length and structure (due to the use of D segments), it also forms a limited number of main-chain conformations for short loop lengths which depend on the length and the presence of particular residues, or types of residue, at key positions in the loop and the antibody framework (Martin et al. (1996) J. Mol. Biol., 263: 800; Shirai et al. (1996) FEBS Letters, 399:1.

Bispecific antibodies comprising complementary pairs of V_{H} and V_{L} regions are known in the art. These bispecific antibodies must comprise two pairs of V_{H} and V_{L}s, each V_{H}/V_{L} pair binding to a single antigen or epitope. Methods described involve hybrid hybridomas (Milstein & Cuello AC, Nature 305:537-40), minibodies (Hu et al., (1996) Cancer Res 56:3055-3061;), diabodies (Holliger et al., (1993) Proc. Natl. Acad. Sci. USA 90, 6444-6448; WO 94/13804), chelating recombinant antibodies (CRAbs; Neri et al., (1995) J. Mol. Biol. 246, 367-373), biscFv (e.g. Atwell et al., (1996) Mol. Immunol. 33, 1301-1312), “knobs in holes” stabilized antibodies (Carter et al., (1997) Protein Sci. 6, 781-788). In each case each antibody species comprises two antigen-binding sites, each fashioned by a complementary pair of V_{H} and V_{L} domains. Each antibody is thereby able to bind to two different antigens or epitopes at the same time, with the binding to EACH antigen or epitope mediated by a V_{H} and its complementary V_{L} domain. Each of these techniques presents its particular disadvantages; for instance in the case of hybrid hybridomas, inactive V_{H}/V_{L} pairs can greatly reduce the fraction of bispecific IgG. Furthermore, most bispecific approaches rely on the association of the different V_{H}/V_{L} pairs or the association of V_{H} and V_{L} chains to recreate the two different V_{H}/V_{L} binding sites. It is therefore impossible to control the ratio of binding sites to each antigen or epitope in the assembled molecule and thus many of the assembled molecules will bind to one antigen or epitope but not the other. In some cases it has been possible to engineer the heavy or light chains at the sub-unit interfaces (Carter et al., 1997) in order to improve the number of molecules which have binding sites to both antigens or epitopes but this never results in all molecules having binding to both antigens or epitopes.

There is some evidence that two different antibody binding specificities might be incorporated into the same binding site, but these generally represent two or more specificities that correspond to structurally related antigens or epitopes or to antibodies that are broadly cross-reactive. For example, cross-reactive antibodies have been described, usually where the two antigens are related in sequence and structure, such as hen egg white lysozyme and turkey lysozyme (McCafferty et al., WO 92/01047) or to free hapten and to hapten conjugated to carrier (Griffiths AD et al. EMBO J 1994 13:14 3245-60). In a further example, WO 02/02773 (Abbott Laboratories) describes antibody molecules with “dual specificity.” The antibody molecules referred to are antibodies raised or selected against multiple antigens, such that their specificity spans more than a single antigen. Each complementary V_{H}/V_{L} pair in the antibodies of WO 02/02773 specifies a single binding specificity for two or more structurally related antigens; the V_{H} and V_{L} domains in such complementary pairs do not each possess a separate specificity. The antibodies thus have a broad single specificity which encompasses two antigens, which are structurally related. Furthermore natural autoantibodies have been described that are polyreactive (Casali & Notkins, Ann. Rev. Immunol. 7, 515-531), reacting with at least two (usually more) different antigens or epitopes that are not structurally related. It has also been shown that selections of random peptide repertoires using phage display technology on a monoclonal antibody will identify a range of peptide sequences that fit the antigen binding site. Some of the sequences are highly related, fitting a consensus sequence, whereas others are very different and have been termed mimotopes (Lane & Stephen, Current Opinion in Immunology, 1993, 5, 268-271). It is therefore clear that a natural four-chain antibody, comprising associated and complementary V_{H} and V_{L} domains, has the potential to bind to many different antigens from a large universe of known antigens. It is less clear how to create a binding site to two given antigens in the same antibody, particularly those which are not necessarily structurally related.

Protein engineering methods have been suggested that may have a bearing on this. For example it has also been proposed that a catalytic antibody could be created with a binding activity to a metal ion through one variable domain, and to a hapten (substrate) through contacts with the metal ion and a complementary variable domain (Barbas et al., 1993 Proc. Natl. Acad. Sci USA 90, 6385-6389). However in this case, the binding and catalysis of the substrate (first antigen) is proposed to require the binding of the metal ion (second antigen). Thus the binding to the V_{H}/V_{L} pairing relates to a single but multi-component antigen.

Methods have been described for the creation of bispecific antibodies from camel antibody heavy chain single domains in which binding contacts for one antigen are created in one variable domain, and for a second antigen in a second immunoglobulin variable domain. However the variable domains were not complementary. Thus a first heavy chain variable domain is selected against a first antigen, and a second heavy chain variable domain against a second antigen, and then both domains are linked together on the same chain to give a bispecific antibody fragment (Conrath et al., J. Biol. Chem. 270, 27589-27594). However the camel heavy chain single domains are unusual in that they are derived from natural camel antibodies which have no light chains, and indeed the heavy chain single domains are unable to associate with camel light chains to form complementary V_{H} and V_{L} pairs.

Single heavy chain variable domains have also been described, derived from natural antibodies which are normally associated with light chains (from monoclonal antibodies or from repertoires of domains; see EP-A-0368684). These heavy chain variable domains have been shown to interact specifically with one or more related antigens but have not been combined with other heavy or light chain variable domains to create a ligand with a specificity for two or more different antigens. Furthermore, these single domains have been shown to have a very short in vivo half-life. Therefore such domains are of limited therapeutic value.

It has been suggested to make bispecific antibody fragments by linking heavy chain variable domains of different specificity together (as described above). The disadvantage with this approach is that isolated antibody variable domains may have a hydrophobic interface that normally makes interactions with the light chain and is exposed to solvent and may be “sticky” allowing the single domain to bind to hydrophobic surfaces. Furthermore, in the absence of a partner light chain the combination of two or more different heavy chain variable domains and their association, possibly via their hydrophobic interfaces, may prevent them from binding to one in not both of the ligands they are able to bind in isolation. Moreover, in this case the heavy chain variable domains would not be associated with complementary light chain variable domains and thus may be less stable and readily unfold (Worn & Pluckthun, 1998 Biochemistry 37, 13120-7).

As used herein, the term “antibody” includes immunoglobulin molecules comprising four polypeptide chains, two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chains of antibodies (immunoglobulins) from any vertebrate species can be assigned to one of two clearly distinct types, called kappa (κ) and lambda (λ), based on the amino acid sequences of their constant domains. The variable regions of kappa light chains are referred to herein as VK. The expression V_{L}, as used herein, is intended to include both the variable regions from kappa-type light chains (VK) and from lambda-type light chains. The light chain constant region is comprised of one domain, CL. The V_{H} and V_{L} regions include regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1-CDR1-FR2-CDR2-FR3-CDR3-FR4.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of antibodies are called α, δ, ε, γ, and μ, respectively. The subunit structures and three-dimensional configurations of different classes of immunoglobulins are well known. The present invention includes antibodies of any of the aforementioned classes or subclasses (isotypes).

The term “antibody” as used herein is also intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof; each containing at least one CDR. Functional fragments include antigen binding fragments that bind to an ADAMTS5 antigen. For example, antibody fragments capable of binding to ADAMTS5or a portion thereof, including, but not limited to Fab (e.g., by papain digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the present invention. Antibody fragments are also intended to include, e.g., domain deleted antibodies, diabodies, linear antibodies, single-chain antibody molecules, and multispecific antibodies formed from antibody fragments.

The term “monoclonal antibody,” as used herein, refers to an antibody obtained from a population of substantially homogeneous antibodies, e.g., the individual antibodies comprising the population are substantially identical except for possible naturally occurring mutations or minor post-translational variations that may be present. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. The monoclonal antibodies of the present invention are preferably made by recombinant DNA methods or are obtained by screening methods as described elsewhere herein.

The term “monoclonal antibodies,” as used herein, includes “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species (e.g., mouse or rat) or belonging to another antibody class or subclass, as well as fragments of such antibodies, so long as they exhibit the desired biological activity (Morrison et al., Proc. Natl. Acad. Sci. USA 81:6851-6855 (1984)). Chimeric antibodies of interest herein include “primatized” antibodies comprising variable domain antigen-binding sequences derived from a non-human primate (e.g., Old World Monkey, such as baboon, rhesus or cynomolgus monkey) and human constant region sequences (U.S. Pat. No. 5,693,780).

Thus, the present invention includes, for example, chimeric monoclonal antibodies comprising a chimeric heavy chain and/or a chimeric light chain. The chimeric heavy chain may comprise any of the heavy chain variable (V_{H}) regions described herein or mutants or variants thereof fused to a heavy chain constant region of a non-human or a human antibody. The chimeric light chain may comprise any of the light chain variable (V_{L}) regions described herein or mutants or variants thereof fused to a light chain constant region of a non-human or a human antibody.

The term “human antibody,” as used herein, includes antibodies having variable and constant regions corresponding to human germline immunoglobulin sequences as described by Kabat et al. (See Kabat, et al. (1991) Sequences of Proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The human antibody can have at least one position replaced with an amino acid residue, e.g., an activity enhancing amino acid residue which is not encoded by the human germline immunoglobulin sequence. In the context of the present invention, the human antibody can have up to twenty positions replaced with amino acid residues which are not part of the human germline immunoglobulin sequence. In other embodiments, up to ten, up to five, up to three or up to two positions are replaced. However, the term “human antibody,” as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal that is transgenic for human immunoglobulin genes, or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). According to the present invention, recombinant human antibodies include human germline immunoglobulin sequence that have been subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo. In certain embodiments, however, such recombinant antibodies are the result of selective mutagenesis approach or backmutation or both.

The antibodies of the present invention may be isolated antibodies. An “isolated antibody,” as used herein, includes an antibody that is substantially free of other antibodies having different antigenic specificities. Moreover, an isolated antibody may be substantially free of other cellular material and/or chemicals.

Intact antibodies include heteromultimeric glycoproteins comprising at least two heavy and two light chains. Aside from IgM, intact antibodies are usually heterotetrameric glycoproteins of approximately 150 Kda, composed of two identical light (L) chains and two identical heavy (H) chains. Typically, each light chain is linked to a heavy chain by one covalent disulfide bond while the number of disulfide linkages between the heavy chains of different immunoglobulin isotypes varies. Each heavy and light chain also has intrachain disulfide bridges. Each heavy chain has at one end a variable domain (V_{H}) followed by a number of constant regions. Each light chain has a variable domain (V_{L}) and a constant region at its other end; the constant region of the light chain is aligned with the first constant region of the heavy chain and the light chain variable domain is aligned with the variable domain of the heavy chain. The light chains of antibodies from most vertebrate species can be assigned to one of two types called Kappa and Lambda based on the amino acid sequence of the constant region. Depending on the amino acid sequence of the constant region of their heavy chains, human antibodies can be assigned to five different classes, IgA, IgD, IgE, IgG and IgM. IgG and IgA can be further subdivided into subclasses, IgG1, IgG2, IgG3 and IgG4; and IgA1 and IgA2. Species variants exist with mouse and rat having at least IgG2a, IgG2b. The variable domain of the antibody confers binding specificity upon the antibody with certain regions displaying particular variability called complementarity determining regions (CDRs). The more conserved portions of the variable region are called Framework regions (FR). The variable domains of intact heavy and light chains each comprise four FR connected by three CDRs. The CDRs in each chain are held together in close proximity by the FR regions and with the CDRs from the other chain contribute to the formation of the antigen binding site of antibodies. The constant regions are not directly involved in the binding of the antibody to the antigen but exhibit various effector functions such as participation in antibody dependent cell-mediated cytotoxicity (ADCC), phagocytosis via binding to Fcγ receptor, half-life/clearance rate via neonatal Fc receptor (FcRn) and complement dependent cytotoxicity via the C1C1q C1qq component of the complement cascade.

Human antibodies may be produced by a number of methods known to those of skill in the art. Human antibodies can be made by the hybridoma method using human myeloma or mouse-human heteromyeloma cells lines see Kozbor J.Immunol 133, 3001, (1984) and Brodeur, Monoclonal Antibody Production Techniques and Applications, pp51-63 (Marcel Dekker Inc, 1987). Alternative methods include the use of phage libraries or transgenic mice both of which utilize human V region repertories (see Winter G, (1994), Annu Rev.Immunol 12,433-455, Green L L (1999), J.Immunol.methods 231, 11-23).

Several strains of transgenic mice are now available wherein their mouse immunoglobulin loci has been replaced with human immunoglobulin gene segments (see Tomizuka K, (2000) PNAS 97,722-727; Fishwild D. M (1996) Nature Biotechnol. 14,845-851, Mendez M J, 1997, Nature Genetics, 15,146-156). Upon antigen challenge such mice are capable of producing a repertoire of human antibodies from which antibodies of interest can be selected. Of particular note is the Trimera™ system (see Eren R et al, (1998) Immunology 93:154-161) where human lymphocytes are transplanted into irradiated mice, the Selected Lymphocyte Antibody System (SLAM, see Babcook et al, PNAS (1996) 93:7843-7848) where human (or other species) lymphocytes are effectively put through a massive pooled in vitro antibody generation procedure followed by deconvulated, limiting dilution and selection procedure and the Xenomouse II™ (Abgenix Inc). An alternative approach is available from Morphotek Inc using the Morphodoma™ technology.

Phage display technology can be used to produce human antibodies (and fragments thereof), see McCafferty; Nature, 348, 552-553 (1990) and Griffiths AD et at (1994) EMBO 13:3245-3260. According to this technique antibody V domain genes are cloned in frame into either a major or minor coat of protein gene of a filamentous bacteriophage such as M13 or fd and displayed (usually with the aid of a helper phage) as functional antibody fragments on the surface of the phage particle. Selections based on the functional properties of the antibody result in selection of the gene encoding the antibody exhibiting those properties. The phage display technique can be used to select antigen specific antibodies from libraries made from human B cells taken from individuals afflicted with a disease or disorder described above or alternatively from unimmunized human donors (see Marks; J. Mol. Bio. 222,581-597, 1991). Where an intact human antibody is desired comprising a Fc domain it is necessary to redone the phage displayed derived fragment into a mammalian expression vectors comprising the desired constant regions and establishing stable expressing cell lines.

The technique of affinity maturation (Marks; Bio/technol 10,779-783 (1992)) may be used to improve binding affinity wherein the affinity of the primary human antibody is improved by sequentially replacing the H and L chain V regions with naturally occurring variants and selecting on the basis of improved binding affinities. Variants of this technique such as “epitope imprinting” are now also available see WO 93/06213. See also Waterhouse; Nucl.Acids Res 21, 2265-2266 (1993).

In certain embodiments, the antigen binding proteins of the present invention have an affinity of at least about 5×10⁴ liter/mole, 1×10⁵ liter/mole, 5×10⁵ liter/mole, or 1×10⁶ liter/mole as measured by an association constant (Ka). In another embodiment, the antigen binding proteins of the present invention binds to a neutralizing epitope of human ADAMTS5 with a dissociation constant (Kd) of less than about 5×10⁻⁴ liter/second, 1×10⁻⁵ liter/second, 5×10⁻⁵ liter/second, or 1×10⁻⁶ liter/second.

The use of intact non-human antibodies in the treatment of human diseases or disorders carries with it the potential for the now well established problems of immunogenicity, which is the immune system of the patient may recognize the non-human intact antibody as non-self and mount a neutralizing response. This reaction is particularly evident upon multiple administration of the non-human antibody to a human patient. Various techniques have been developed over the years to overcome these problems and generally involve reducing the composition of non-human amino acid sequences in the intact antibody whilst retaining the relative ease in obtaining non-human antibodies from an immunized animal e.g. mouse, rat or rabbit. Broadly two approaches have been used to achieve this. The first are chimeric antibodies, which generally comprise a non-human (e.g. rodent such as mouse) variable domain fused to a human constant region. Because the antigen-binding site of an antibody is localized within the variable regions the chimeric antibody retains its binding affinity for the antigen but acquires the effector functions of the human constant region and are therefore able to perform effector functions such as described supra. Chimeric antibodies are typically produced using recombinant DNA methods. DNA encoding the antibodies (e.g. cDNA) is isolated and sequenced using conventional procedures (e.g. by using oligonucleotide probes that are capable of binding specifically to genes encoding the H and L chains of the antibody of the invention, e.g. DNA encoding SEQ.I.D.NO 2, 3, 4, 5, 6 and 7 described supra). Hybridoma cells serve as a typical source of such DNA. Once isolated, the DNA is placed into expression vectors which are then transfected into host cells such as E. coli, COS cells, CHO cells or myeloma cells that do not otherwise produce immunoglobulin protein to obtain synthesis of the antibody. The DNA may be modified by substituting the coding sequence for human L and H chains for the corresponding non-human (e.g. murine) H and L constant regions see e.g. Morrison; PNAS 81, 6851 (1984).

The second approach involves the generation of humanized antibodies wherein the non-human content of the antibody is reduced by humanizing the variable regions. Humanized antibodies can be made by CDR grafting. CDRs build loops close to the antibody's N-terminus where they form a surface mounted in a scaffold provided by the framework regions. Antigen-binding specificity of the antibody is mainly defined by the topography and by the chemical characteristics of its CDR surface. These features are in turn determined by the conformation of the individual CDRs, by the relative disposition of the CDRs, and by the nature and disposition of the side chains of the residues comprising the CDRs. A large decrease in immunogenicity can be achieved by grafting only the CDRs of a non-human (e.g. murine) antibodies (“donor” antibodies) onto human framework (“acceptor framework”) and constant regions (see Jones et at (1986) Nature 321,522-525 and Verhoeyen M et at (1988) Science 239, 1534-1536). However, CDR grafting per se may not result in the complete retention of antigen-binding properties and it is frequently found that some framework residues (sometimes referred to as “back-mutations”) of the donor antibody need to be preserved in the humanized molecule if significant antigen-binding affinity is to be recovered (see Queen C et at (1989) PNAS 86, 10,029-10,033, Co, M et al (1991) Nature 351, 501-502). In this case, human V regions showing the greatest sequence homology to the non-human donor antibody are chosen from a database in order to provide the human framework (FR). The selection of human FRs can be made either from human consensus or individual human antibodies. Where necessary key residues from the donor antibody are substituted into the human acceptor framework to preserve CDR conformations. Computer modeling of the antibody maybe used to help identify such structurally important residues, see WO99/48523.

Alternatively, humanization may be achieved by a process of “veneering.” A statistical analysis of unique human and murine immunoglobulin heavy and light chain variable regions revealed that the precise patterns of exposed residues are different in human and murine antibodies, and most individual surface positions have a strong preference for a small number of different residues (see Padlan E. A. et al; (1991) Mol.Immuno1.28, 489-498 and Pedersen J. T. et at (1994) J.Mol.Biol. 235; 959-973). Therefore it is possible to reduce the immunogenicity of a non-human Fv by replacing exposed residues in its framework regions that differ from those usually found in human antibodies. Because protein antigenicity may be correlated with surface accessibility, replacement of the surface residues may be sufficient to render the mouse variable region “invisible” to the human immune system (see also Mark G. E. et at (1994) in Handbook of Experimental Pharmacology vol.113: The pharmacology of monoclonal Antibodies, Springer-Verlag, pp105-134). This procedure of humanization is referred to as “veneering” because only the surface of the antibody is altered, the supporting residues remain undisturbed.

Thus, the present invention provides isolated antigen binding proteins, comprising at least one first immunoglobulin variable domain capable of binding to an aggrecanase. In one embodiment, the aggrecanase is human ADAMTS5. In some instances, the antigen binding protein is an antibody or fragment thereof. In some instances the antibody specifically binds to ADAMTS5. The antibody may be a monoclonal antibody or fragment thereof. In some instances, the monoclonal antibodies or fragment thereof of the present invention are mouse, chimeric, humanized, or fully human.

In another embodiment, the antigen binding protein comprises at least one complementarity determining region. In some instances, the antigen binding protein of the present invention is a monoclonal antibody comprising a heavy chain comprising CDRH1, CDRH2 and CDRH3 and a light chain comprising CDRL1, CDRL2 and CDRL3, wherein the complementarity determining regions (CDRs) of the heavy chain are selected from the group of:
CDRH1 having at least about 80% sequence identity to amino acid sequence

(SEQ ID NO: 2)
DAWMD;

CDRH2 having at least about 70, 75, 80, 85, 90, 95, or 98% sequence identity to amino acid sequence EIRHKANDHAIFYXESVKG (SEQ ID NO:3); and
CDRH3 having at least about 70, 75, 80, 85, 90, 95, or 98% sequence identity to amino acid sequence TYYYGSSYGYCDV (SEQ ID NO:4) or PFAY (SEQ ID NO:5); and

the complementarity determining regions of the light chain are selected from the group of:
CDRL1 having at least about 70, 75, 80, 85, 90, 95, or 98% sequence identity to amino acid sequence KASQSVGTTIV (SEQ ID NO:6) or RTSENIYSYLA (SEQ ID NO:7);
CDRL2 having at least about 70, 75, 80, 85, 90, 95, or 98% sequence identity to amino acid sequence NAKTLAE (SEQ ID NO:8) or SASNRXT (SEQ ID NO:9) ; and
CDRL3 having at least about 70, 75, 80, 85, 90, 95, or 98% sequence identity to amino acid sequence QQYSSYPFT(SEQ ID NO:10) or QHHYGTPWT ((SEQ ID NO:11).

In one embodiment, CDRH2 has at least about 70, 75, 80, 85, 90, 95, or 98% sequence identity an amino acid sequence selected from EIRHKANDHAIFYAESVKG (SEQ ID NO:12), EIRNKANNHARHYAESVKG (SEQ ID NO:13), EIRHKANDYAIFYDESVKG (SEQ ID NO:14), EIRHKANDHAIFYDESVKG (SEQ ID NO:15), DIRNTANNHATFYAESVKG (SEQ ID NO:16), and EIRHKANDHAIFYDESVKG (SEQ ID NO:17). In one embodiment, CDRH3 comprises the amino acid sequence, PFAY (SEQ ID NO:5).

In yet another embodiment, the antigen binding proteins of the present invention are monoclonal antibodies comprising a heavy chain comprising CDRH1, CDRH2 and CDRH3 and a light chain comprising CDRL1, CDRL2 and CDRL3, wherein the complementarity determining regions (CDRs) of the heavy chain are selected from:
CDRH1 is amino acid sequence DAWMD (SEQ ID NO:2);
CDRH2 is select from amino acid sequence

(SEQ ID NO: 12)
EIRHKANDHAIFYAESVKG,
(SEQ ID NO: 13)
EIRNKANNHARHYAESVKG,
(SEQ ID NO: 14)
EIRHKANDYAIFYDESVKG,
(SEQ ID NO: 15)
EIRHKANDHAIFYDESVKG,
(SEQ ID NO: 16)
DIRNTANNHATFYAESVKG,
or
(SEQ ID NO:17)
EIRHKANDHAIFYDESVKG;
and
(SEQ ID NO: 18)
CDRH3 is TYYYGSSYGYCDV
or
(SEQ ID NO: 5)
PFAY;
and

the complementarity determining regions of the light chain are selected from:
CDRL1 is select from amino acid sequence KASQSVGTTIV (SEQ ID NO:19), RTSENIYSYLA (SEQ ID NO:20), or KASQNVGTAVV (SEQ ID NO:21);
CDRL2 is select from amino acid sequence NAKTLAE (SEQ ID NO:22), SASNRHT (SEQ ID NO:23), SASTRYT (SEQ ID NO:24), or SASNRYT (SEQ ID NO:25); and
CDRL3 is select from amino acid sequence QQYSSYPFT (SEQ ID NO:26), QHHYGTPWT (SEQ ID NO:27), QQYVNYPFT (SEQ ID NO:28), or QQYTSYPFT (SEQ ID NO:29).

Thus, in one embodiment of the present invention, an isolated monoclonal antibody is provided comprising six CDRs wherein CDRH1 is DAWMD (SEQ ID NO:2), CDRH2 is EIRNKANNHARHYAESVKG (SEQ ID NO:13), and CDRH3 is TYYYGSSYGYCDV (SEQ ID NO:18) and CDRL1 is RTSENIYSYLA (SEQ ID NO:20), CDRL2 is NAKTLAE (SEQ ID NO:22) and CDRL3 is QHHYGTPWT (SEQ ID NO:27). In another embodiment of the present invention, an isolated monoclonal antibody is provided comprising six CDRs wherein CDRH1 is DAWMD (SEQ ID NO:2), CDRH2 is EIRHKANDHAIFYDESVKG (SEQ ID NO:15), and CDRH3 is PFAY (SEQ ID NO:5) and CDRL1 is KASQSVGTTIV (SEQ ID NO:19), CDRL2 is SASNRHT (SEQ ID NO:23) and CDRL3 is QQYTSYPFT (SEQ ID NO:29).

In yet another embodiment, the antigen binding proteins of the present invention are monoclonal antibodies comprising a heavy chain comprising CDRH1, CDRH2 and CDRH3 and a light chain comprising CDRL1, CDRL2 and CDRL3, wherein the complementarity determining regions (CDRs) of the heavy chain are selected from:
CDRH1 is amino acid sequence DAWMD (SEQ ID NO:2), wherein any amino acid of SEQ ID NO: 2 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine;
CDRH2 is select from amino acid sequence EIRHKANDHAIFYAESVKG (SEQ ID NO:12), EIRNKANNHARHYAESVKG (SEQ ID NO:13),
EIRHKANDYAIFYDESVKG (SEQ ID NO:14),
EIRHKANDHAIFYDESVKG (SEQ ID NO:15),
DIRNTANNHATFYAESVKG (SEQ ID NO:16), or
EIRHKANDHAIFYDESVKG (SEQ ID NO:17) , wherein any amino acid of SEQ ID NOS: 12-17 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine; and
CDRH3 is TYYYGSSYGYCDV (SEQ ID NO:18) or
PFAY (SEQ ID NO:5) , wherein any amino acid of SEQ ID NOS: 18 and 5 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine; and the complementarity determining regions of the light chain are selected from:
CDRL1 is select from amino acid sequence KASQSVGTTIV (SEQ ID NO:19), RTSENIYSYLA (SEQ ID NO:20), or KASQNVGTAVV (SEQ ID NO:21) , wherein any amino acid of SEQ ID NO: 19-21 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine;
CDRL2 is select from amino acid sequence NAKTLAE (SEQ ID NO:22), SASNRHT (SEQ ID NO:23), SASTRYT (SEQ ID NO:24), or SASNRYT (SEQ ID NO:25) , wherein any amino acid of SEQ ID NO: 22-25 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine; and CDRL3 is select from amino acid sequence QQYSSYPFT (SEQ ID NO:26), QHHYGTPWT (SEQ ID NO:27), QQYVNYPFT (SEQ ID NO:28), or QQYTSYPFT (SEQ ID NO:29), wherein any amino acid of SEQ ID NO: 26-29 is substituted at one position by an amino acid selected from histidine, isoleucine, leucine, lysine, methionine, phenylalanine, threonine, tryptophan, valine, alanine, arginine, aspartic acid, cysteine, cystine, glutamic acid, glutamine, glycine, ornithine, proline, serine, taurine, and tyrosine.

In certain embodiments, Thr4 of NAKTLAE (SEQ ID NO:22) is leucine, isoleucine or methionine. In certain embodiments, His3 of QHHYGTPWT (SEQ ID NO:27) is valine. In certain embodiments, Gly5 of QHHYGTPWT (SEQ ID NO:27) is tryptophan, tyrosine, phenylalanine, or methionine. In certain embodiments, His9 of EIRNKANNHARHYAESVKG (SEQ ID NO:13) is phenylalanine or tyrosine. In certain embodiments, Ser6 of TYYYGSSYGYCDV (SEQ ID NO:18) is phenylalanine or tyrosine.

The CDRs L1, L2, L3, H1 and H2 tend to structurally exhibit one of a finite number of main chain conformations. The particular canonical structure class of a CDR is defined by both the length of the CDR and by the loop packing, determined by residues located at key positions in both the CDRs and the framework regions (structurally determining residues or SDRs). Martin and Thornton (1996; J Mol Biol 263:800-815) have generated an automatic method to define the “key residue” canonical templates. Cluster analysis is used to define the canonical classes for sets of CDRs, and canonical templates are then identified by analysing buried hydrophobics, hydrogen-bonding residues, and conserved glycines and prolines. The CDRs of antibody sequences can be assigned to canonical classes by comparing the sequences to the key residue templates and scoring each template using identity or similarity matrices.

Examples of CDR canonicals within the scope of the invention are given below. The amino acid numbering used is Kabat.

Examples of canonicals for CDRH1 as set out in SEQ ID NO:144, or a variant thereof are: Ala 32 is substituted for Ile, His, Tyr, Phe, Thr, Asn, Cys, Glu or Asp; Trp 33 is substituted for Tyr, Ala, Gly, Thr, Leu or Val; Met 34 is substituted for Ile, Val or Trp; and Asp 35 is substituted for His, Glu, Asn, Gln, Ser, Tyr or Thr.

Examples of canonicals for CDRH2 as set out in SEQ ID NO:144, or a variant thereof are: Glu 50 is substituted for Arg or Gln; and Ile 51 is substituted for Leu, Val, Thr, Ser or Asn.,

Examples of canonicals for CDRH3 as set out in SEQ ID NO:144, or a variant thereof are: Tyr 102 is substituted for His, Val, Ile, Ser, Asp or Gly.

Examples of canonicals for CDRL1 as set out in SEQ ID NO:146, or a variant thereof are: Ser 28 is substituted for Asn, Asp, Thr or Glu; Val 29 is substituted for Ile; Gly 30 is substituted for Asp, Leu, Tyr, Val, Ile, Ser, Asn, Phe, His or Thr; Thr 31 is substituted for Ser, Asn, Lys or Gly; Thr 32 is substituted for Phe, Tyr, Asn, Ala, His, Ser or Arg; Ile 33 is substituted for Met, Leu, Val or Phe; and Val 34 is substituted for Ala, Gly, Asn, Ser, His or Phe.

Examples of canonicals for CDRL3 as set out in SEQ ID NO:146, or a variant thereof are: Gln 89 is substituted for Ser, Gly, Phe or Leu; Gln 90 is substituted for Asn or His; Tyr 91 is substituted for Asn, Phe, Gly, Ser, Arg, Asp, His, Thr or Val; Thr 92 is substituted for Asn, Tyr, Trp, Ser, Arg, Gln, His, Ala or Asp; Ser 93 is substituted for Gly, Asn, Thr, Arg, Glu, Ala or His; Tyr 94 is substituted for Asp, Thr, Val, Leu, His, Asn, Ile, Tip, Pro or Ser; and Phe 96 is substituted for Pro, Leu, Tyr, Arg, Ile or Trp.

In other aspects the antigen binding protein is a Fab or F(ab)₂ fragment. In another embodiment, the first immunoglobulin variable domain is a single chain variable domain.

In one embodiment of the present invention there is provided an antibody according to the invention described herein and comprising a constant domain region such that the antibody has reduced ADCC and/or complement activation or effector functionality. In one such embodiment the constant domain may comprise a naturally disabled constant region of IgG2 or IgG4 isotype or a mutated IgG1 constant domain. Examples of suitable modifications are described in EP0307434. One example comprises the substitutions of alanine residues at positions 235 and 237 (EU index numbering). In one embodiment, such an antibody comprises the heavy chain of SEQ ID NO:158.

In one embodiment the antigen binding protein or a fragment thereof comprises an antibody V_{H} domain comprising an amino acid sequence selected from the group consisting of SEQ ID NO: 76, 80, 116, 118, 120, 122, 124, 126, 128, 136, 138, 140, 142, and 144.

In one embodiment the antigen binding protein or a fragment thereof comprises an antibody V_{L} domain comprising an amino acid sequence selected from the group consisting of SEQ ID NO: 78, 82, 130, 132, 134, and 146.

In one embodiment the antigen binding protein or a fragment thereof comprises an antibody V_{H} domain comprising an amino acid sequence selected from the group consisting of SEQ ID NO: 76, 80, 116, 118, 120, 122, 124, 126, 128, 136, 138, 140, 142, and 144 and a V_{L} domain comprising an amino acid sequence selected from the group consisting of SEQ ID NO: 78, 82, 130, 132, 134, and 146.

In one embodiment the antigen binding protein or a fragment thereof comprises an antibody V_{H} domain comprising SEQ ID NO: 76 and a V_{L} domain comprising SEQ ID NO: 78.

In one embodiment the antigen binding protein or a fragment thereof comprises an antibody V_{H} domain comprising SEQ ID NO: 80 and a V_{L} domain comprising SEQ ID NO: 82.

In one embodiment the antigen binding protein or a fragment thereof comprises an antibody V_{H} domain comprising an amino acid sequence selected from the group consisting of SEQ ID NO: 116, 118, 120, 122, 124, 126, and 128 and a V_{L} domain comprising an amino acid sequence selected from the group consisting of SEQ ID NO: 130, 132, and 134.

In one embodiment the antigen binding protein or a fragment thereof comprises an antibody V_{H} domain comprising an amino acid sequence selected from the group consisting of SEQ ID NO: 136, 138, 140, 142, and 144 and a V_{L} domain comprising SEQ ID NO: 146.

In one embodiment the antigen binding protein or a fragment thereof comprises an antibody heavy chain comprising an amino acid sequence selected from the group consisting of SEQ ID NO: 68, 72, 84, 86, 88, 90, 92, 94, 96, 104, 106, 108, 110, 112, and 158.

In one embodiment the antigen binding protein or a fragment thereof comprises an antibody light chain comprising an amino acid sequence selected from the group consisting of SEQ ID NO: 70, 74, 98, 100, 102, and 114.

In one embodiment the antigen binding protein or a fragment thereof comprises an antibody heavy chain comprising an amino acid sequence selected from the group consisting of SEQ ID NO: 68, 72, 84, 86, 88, 90, 92, 94, 96, 104, 106, 108, 110, 112, and 158 and an antibody light chain comprising an amino acid sequence selected from the group consisting of SEQ ID NO: 70, 74, 98, 100, 102, and 114.

In one embodiment the antigen binding protein or a fragment thereof comprises an antibody heavy chain comprising SEQ ID NO: 68 and an antibody light chain comprising SEQ ID NO: 70.

In one embodiment the antigen binding protein or a fragment thereof comprises an antibody heavy chain comprising SEQ ID NO: 72 and an antibody light chain comprising SEQ ID NO: 74.

In one embodiment the antigen binding protein or a fragment thereof comprises an antibody heavy chain comprising an amino acid sequence selected from the group consisting of SEQ ID NO: 84, 86, 88, 90, 92, 94, and 96 and an antibody light chain comprising an amino acid sequence selected from the group consisting of SEQ ID NO: 98, 100, and 102.

In one embodiment the antigen binding protein or a fragment thereof comprises an antibody heavy chain comprising an amino acid sequence selected from the group consisting of SEQ ID NO: 104, 106, 108, 110, 112, and 158 and an antibody light chain comprising SEQ ID NO: 114.

Another aspect of the invention includes an antibody that competes for binding to ADAMTS5 with any one of the antibodies listed in Table 3. These include the antibodies 1G10.1C9, 2D3.1D4, 3A12.1D7, 5F10.1H6, 11F12.1D12, 12F4.1H7, and 7B4.1E11.

In yet another embodiment, the antigen binding protein further comprises a second immunoglobulin variable domain, capable of binding to a second antigen. The second immunoglobulin variable domain may bind to an antigen which may act as a carrier upon administration of the antigen binding peptide. For instance, the second immunoglobulin variable domain may bind a blood protein such as, but not limited to, human serum albumin or transferrin. Additionally, the second immunoglobulin variable domain may bind to an antigen associated with modulation of pain in a mammal such as, but not limited to, nerve growth factor (NGF), vasoactive intestinal peptide (VIP), and/or TRPV1 or other vannilloid receptor. Additionally, the second immunoglobulin variable domain may bind to a cytokine or cytokine receptor associated with inflammatory response and/or autoimmune disease such as, but not limited to, oncostatin M (OSM), TNF-α, IL-6, TRPV4, RANKL and IL-1. The second antigen may also bind to a second aggrecanase, such as, but not limited to ADAMTS4 and/or ADAMTS5, and/or a second epitope on ADAMTS5 and/or a number of metalloproteases such as but not limited to MMP-13. The second immunoglobulin variable domain may also bind to aggrecan, collagen II, proteoglycan or other molecules associated with cartilage. In one aspect of the present invention, the second immunoglobulin variable domain binds to one an antigen selected from the group of: human serum albumin, ADAMTS4, NGF, OSM, TNF-α, IL-6, VIP, TRPV1, TRPV4, ADAMTS1, aggrecan, Collagen II, RANKL, Syndecan 4, Hedgehog, and/or IL-1.

Delgado, et al. Nature Med. 7, 563-568 report that VIP treatment suppresses production of pro-inflammatory mediators, as well as expression of the metalloproteinase gelatinase (MMP-2). MMP-2 is believed to contribute to joint destruction in paws of arthritic mice. In vitro studies indicate that VIP may act directly on synoviocytes, although an indirect action could also be mediated through enhanced production of Th2 cytokines Nonetheless, VIP appears to affect synovial function at multiple levels in the CIA mouse model. The peptide suppresses Thl function and but increases Th2 function, possibly ‘rebalancing’ the immune system. Moreover, VIP has direct and indirect effects on macrophages and synoviocytes, leading to decreased expression of IL-1, TNF-α, chemokines and matrix-degrading enzymes, protecting joint integrity. Firestein Nature Medicine 7, 537 - 538 (2001).

Vasoactive intestinal peptide (VIP) was identified in the synovial fluid of arthritis patients nearly 20 years ago and the aim of this study was to examine whether VIP could be involved in the generation of OA pain. Hindlimb weight bearing was used as a measure of joint pain, while von Frey hair algesiometry applied to the plantar surface of the ipsilateral hindpaw tested for secondary mechanical hyperalgesia. Intra-articular injection of VIP into normal rat knee joints caused a significant shift in weight bearing in favor of the contralateral non-injected hindlimb as well as causing a reduction in ipsilateral paw withdrawal threshold. These pain responses were blocked by co-administration of a VPAC receptor antagonist. Antagonists that inhibit VIP activity may prove beneficial in the alleviation of OA pain. McDougall, et al. Pain 2006 Jul;123(1-2):98-105.

Nerve growth factor (NGF) was the first neurotrophin to be identified, and its role in the development and survival of both peripheral and central neurons has been well characterized. NGF has been shown to be a critical survival and maintenance factor in the development of peripheral sympathetic and embryonic sensory neurons and of basal forebrain cholinergic neurons. Smeyne et al., Nature 368:246-249 (1994) and Crowley et al., Cell 76:1001-1011 (1994). NGF up-regulates expression of neuropeptides in sensory neurons (Lindsay and Harmer, Nature 337:362-364 (1989)) and its activity is mediated through two different membrane-bound receptors, the TrkA receptor and the p75 common neurotrophin receptor (sometimes termed “high affinity” and “low affinity” NGF receptors, respectively). Chao et al., Science 232:518-521 (1986). For review on NGF, see Huang et al., Annu Rev. Neurosci. 24:677-736 (2001); Bibel et al., Genes Dev. 14:2919-2937 (2000). The crystal structure of NGF and NGF in complex with the trkA receptor have been determined. See Nature 254:411 (1991); Nature 401:184-188 (1996).

Oncostatin M is a 28 KDa glycoprotein that belongs to the interleukin 6 (IL-6) family of cytokines which includes IL-6, Leukaemia Inhibitory Factor (LIF), ciliary neurotrophic factor (CNTF), cardiotropin-1 (CT-1) and cardiotrophin-1 like cytokine (See Kishimoto T et al (1995) Blood 86: 1243-1254), which share the gp130 transmembrane signalling receptor (See Taga T and Kishimoto T (1997) Annu Rev. Immunol. 15: 797-819). OSM was originally discovered by its ability to inhibit the growth of the melanoma cell line A375 (See Malik N (1989) et al Mol Cell Biol 9: 2847-2853). Subsequently, more effects were discovered and it was found to be a multifunctional mediator like other members of the IL-6 family. OSM is produced in a variety of cell types including macrophages, activated T cells (See Zarling J M (1986) PNAS (USA) 83: 9739-9743), polymorphonuclear neutrophils (See Grenier A et al (1999) Blood 93:1413-1421), eosinophils (See Tamura S et al (2002) Dev. Dyn. 225: 327-31), dendritic cells (See Suda T et al (2002) Cytokine 17:335-340). It is also expressed in pancreas, kidney, testes, spleen stomach and brain (See Znoyko I et al (2005) Anat Rec A Discov Mol Cell Evol Biol 283: 182-186), and bone marrow (See Psenak 0 et al (2003) Acta Haematol 109: 68-75) Its principle biological effects include activation of endothelium (See Brown T J et al (1993) Blood 82: 33-7), activation of the acute phase response (See Benigni F et al (1996) Blood 87: 1851-1854), induction of cellular proliferation or differentiation, modulation of inflammatory mediator release and haematopoesis (See Tanaka M et al (2003) 102: 3154-3162), re-modelling of bone (See de Hooge ASK (2002) Am J Pathol 160: 1733-1743) and, promotion of angiogenesis (See Vasse M et al (1999) Arterioscler Thromb Vasc Biol 19:1835-1842) and wound healing.

The cytokine known as tumor necrosis factor-α (TNFα; also termed cachectin) is a protein secreted primarily by monocytes and macrophages in response to endotoxin or other stimuli as a soluble homotrimer of 17 kD protein subunits (Smith, R. A. et al., J. Biol. Chem. 262:6951-6954 (1987)). A membrane-bound 26 kD precursor form of TNF has also been described (Kriegler, M. et al., Cell 53:45-53 (1988)).

Accumulating evidence indicates that TNF is a regulatory cytokine with pleiotropic biological activities. These activities include: inhibition of lipoprotein lipase synthesis (“cachectin” activity) (Beutler, B. et al., Nature 316:552 (1985)), activation of polymorphonuclear leukocytes (Klebanoff, S. J. et al., J. Immunol. 136:4220 (1986); Perussia, B., et al., J. Immunol. 138:765 (1987)), inhibition of cell growth or stimulation of cell growth (Vilcek, J. et al., J. Exp. Med. 163:632 (1986); Sugarman, B. J. et al., Science 230:943 (1985); Lachman, L. B. et al., J. Immunol. 138:2913 (1987)), cytotoxic action on certain transformed cell types (Lachman, L. B. et al., supra; Darzynkiewicz, Z. et al., Canc. Res. 44:83 (1984)), antiviral activity (Kohase, M. et al., Cell 45:659 (1986); Wong, G. H. W. et al., Nature 323:819 (1986)), stimulation of bone resorption (Bertolini, D. R. et al., Nature 319:516 (1986); Saklatvala, J., Nature 322:547 (1986)), stimulation of collagenase and prostaglandin E2 production (Dayer, J.-M. et al., J. Exp. Med. 162:2163 (1985)); and immunoregulatory actions, including activation of T cells (Yokota, S. et al., J. Immunol. 140:531 (1988)), B cells (Kehrl, J. H. et al., J. Exp. Med. 166:786 (1987)), monocytes (Philip, R. et al., Nature 323:86 (1986)), thymocytes (Ranges, G. E. et al., J. Exp. Med. 167:1472 (1988)), and stimulation of the cell-surface expression of major histocompatibility complex (MHC) class I and class II molecules (Collins, T. et al., Proc. Natl. Acad. Sci. USA 83:446 (1986); Pujol-Borrel, R. et al., Nature 326:304 (1987)).

Interleukin-6 (IL-6) is a 22 to 27 kDa secreted glycoprotein which exhibits growth stimulatory and proimflammatory activities. IL-6 is also known as interferon-β2 (IFN-Jβ2), IL-1 inducible 26-kDa protein, hepatocyte-stimulating factor, cytotoxic T-cell differentiation factor, and B-cell stimulatory factor. (Trikha et al., Clin. Cancer Res. 9:4653-4665 (2003)). IL-6 is secreted by various cell types. IL-6 exerts its activities through binding to a high-affinity receptor complex consisting of two membrane glycoproteins: an 80 kDa component receptor that binds IL-6 with low affinity (IL-6R) and a signal-transducing component of 130 kDa (gp130) that does not bind IL-6 by itself, but is required for high-affinity binding of IL-6 by the complex. IL-6R can be cleaved by a transmembrane metalloproteinase to yield the soluble IL-6R.

RANK is a member of the TNF receptor superfamily; it most closely resembles CD40 in the extracellular region. Similar to CD40, RANK associates with TRAF2 and TRAF3 (as determined by co-immunoprecipitation assays substantially as described by Rothe et al., Cell 83:1243, 1995). TRAFs are critically important in the regulation of the immune and inflammatory response. Through their association with various members of the TNF receptor superfamily, a signal is transduced to a cell. That signal results in the proliferation, differentiation or apoptosis of the cell, depending on which receptor(s) is/are triggered and which TRAF(s) associate with the receptor(s); different signals can be transduced to a cell via coordination of various signaling events. Thus, a signal transduced through one member of this family may be proliferative, differentiative or apoptotic, depending on other signals being transduced to the cell, and/or the state of differentiation of the cell. Such exquisite regulation of this proliferative/apoptotic pathway is necessary to develop and maintain protection against pathogens; imbalances can result in autoimmune disease.

RANK is expressed on epithelial cells, some B cell lines, and on activated T cells. However, its expression on activated T cells is late, about four days after activation. This time course of expression coincides with the expression of Fas, a known agent of apoptosis. RANK may act as an anti-apoptotic signal, rescuing cells that express RANK from apoptosis as CD40 is known to do. Alternatively, RANK may confirm an apoptotic signal under the appropriate circumstances, again similar to CD40. RANK and its ligand are likely to play an integral role in regulation of the immune and inflammatory response.

The ligand, which is referred to as RANKL, is a Type 2 transmembrane protein with an intracellular domain of less than about 50 amino acids, a transmembrane domain and an extracellular domain of from about 240 to 250 amino acids. Similar to other members of the TNF family to which it belongs, RANKL has a ‘spacer’ region between the transmembrane domain and the receptor binding domain that is not necessary for receptor binding. Accordingly, soluble forms of RANKL can comprise the entire extracellular domain or fragments thereof that include the receptor binding region.

TRPV4 channel receptor is one of six known members of the vanilloid family of transient receptor potential channels and shares 51% identity at the nucleotide level with TRPV 1, the capsaicin receptor. Examples of polypeptides and polynucleotides encoding forms of human vanniloid receptors, including TRPV4 channel receptor from human, can be found in EP 1170365 as well as WO 00/32766. Like the other family members, TRPV4 channel receptor is a Ca2+ permeable, non-selective, ligand-gated cation channel, which is responsive to diverse stimuli such as reduced osmolality, elevated temperature, and small molecule ligands. See, for instance, Voets, et al., J. Biol. Chem. (2002) 277 33704-47051; Watanabe, et al., J. Biol. Chem. (2002) 277:47044-47051; Watanabe, et al., J. Biol. Chem. (2002) 277: 13569-47051; Xu, et al., J. Biol. Chem. (2003) 278:11520-11527. From a screen of body tissues, the human TRPV4 channel receptor is most prominently expressed in cartilage. A screen of primary and clonal cell cultures shows significant expression only in chondrocytes.

Such responses are also evoked by structural analogues of capsaicin that share a common vanilloid moiety. One such analogue is resiniferatoxin (RTX), a natural product of Euphorbia plants. The term vanilloid receptor (VR) was coined to describe the neuronal membrane recognition site for capsaicin and such related irritant compounds. The capsaicin response is competitively inhibited (and thereby antagonized) by another capsaicin analog, capsazepine, and is also inhibited by the non-selective cation channel blocker ruthenium red. These antagonists bind to VR with no more than moderate affinity (typically with Kᵢ values of no lower than 140 μM).

Recently, rat and human receptors for capsaicin were cloned from dorsal root ganglion cells. Such receptors have also been referred to as VR1, and the terms “VR1” and “capsaicin receptor” are used interchangeably herein to refer to rat and/or human receptors of this type, as well as mammalian homologs. The role of VR1 in pain sensation has been confirmed using mice lacking this receptor, which exhibit no vanilloid-evoked pain behavior, and impaired responses to heat and inflammation. The capsaicin receptor is a nonselective cation channel with a threshold for opening that is lowered in response to elevated temperatures, low pH, and capsaicin receptor agonists. For example, the channel usually opens at temperatures higher than about 45° C. Opening of the capsaicin receptor channel is generally followed by the release of inflammatory peptides from neurons expressing the receptor and other nearby neurons, increasing the pain response. After initial activation by capsaicin, the capsaicin receptor undergoes a rapid desensitization via phosphorylation by cAMP-dependent protein kinase.

The antigen binding protein of the present invention can be characterized by a dissociation constant equal or less than about 9.0×10⁻⁹ M for human ADAMTS5, in some instances it is less than or equal to about 2.5×10⁻¹⁰ M. Antigen binding protein affinity for a target such as human ADAMTS5 can be measured by surface plasmon resonance such as but not limited to BIACORE or Octet. BIAcore kinetic analysis can be used to determine the binding on and off rates of antibodies or fragments thereof to a ADAMTS5 antigen. BIAcore kinetic analysis comprises analyzing the binding and dissociation of a ADAMTS5 antigen from chips with immobilized antibodies or fragments thereof on their surface (see the Example section infra).

The present invention also provides antigen binding proteins that block and/or reduce at least one activity ADAMTS5. In some instances, the antigen binding proteins of the present invention blocks and/or reduces the cleavage of aggrecan by ADAMTS5 at the Glu³⁷³-Ala³⁷⁴ cleavage site. In some aspects, the antigen binding proteins of the present invention are capable of penetrating cartilage, even when administered by a non-articular route of administration. For instance, the antigen binding proteins of the present invention may be administered intravenously, intramuscularly, intraarticularly, subcutaneously, orally, intranasally, and/or by peritoneal administration.

Also provided in the present invention are isolated polynucleotides encoding an antigen binding protein of this invention.

In one embodiment the isolated polynucleotide encodes an antigen binding protein or a fragment thereof comprising an antibody V_{H} domain comprising an amino acid sequence selected from the group consisting of SEQ ID NO: 76, 80, 116, 118, 120, 122, 124, 126, 128, 136, 138, 140, 142, and 144. In one embodiment the isolated polynucleotide is selected from the group consisting of SEQ ID NO: 75, 79, 115, 117, 119, 121, 123, 125, 127, 135, 137, 139, 141, 143, and 159. In one embodiment the polypeptide is an antibody produced from a cell expressing a polynucleotide selected from the group consisting of SEQ ID NO: 75, 79, 115, 117, 119, 121, 123, 125, 127, 135, 137, 139, 141, 143, and 159.

In one embodiment the isolated polynucleotide encodes an antigen binding protein or a fragment thereof comprising an antibody V_{L} domain comprising an amino acid sequence selected from the group consisting of SEQ ID NO: 78, 82, 130, 132, 134, and 146. In one embodiment the isolated polynucleotide is selected from the group consisting of SEQ ID NO: 77, 81, 129, 131, 133, and 145. In one embodiment the polypeptide is an antibody produced from a cell expressing a polynucleotide selected from the group consisting of SEQ ID NO: 77, 81, 129, 131, 133, and 145.

In one embodiment the isolated polynucleotide encodes an antigen binding protein or a fragment thereof comprising an antibody heavy chain comprising an amino acid sequence selected from the group consisting of SEQ ID NO: 68, 72, 84, 86, 88, 90, 92, 94, 96, 104, 106, 108, 110, 112, and 158. In one embodiment the isolated polynucleotide is selected from the group consisting of SEQ ID NO: 67, 71, 83, 85, 87, 89, 91, 93, 95, 103, 105, 107, 109, 111, and 159. In one embodiment the polypeptide is an antibody produced from a cell expressing a polynucleotide selected from the group consisting of SEQ ID NO: 67, 71, 83, 85, 87, 89, 91, 93, 95, 103, 105, 107, 109, 111, and 159.

In one embodiment the isolated polynucleotide encodes an antigen binding protein or a fragment thereof comprising an antibody light chain comprising an amino acid sequence selected from the group consisting of SEQ ID NO: 70, 74, 98, 100, 102, and 114. In one embodiment the isolated polynucleotide is selected from the group consisting of SEQ ID NO: 69, 73, 97, 99, 101, and 115. In one embodiment the polypeptide is an antibody produced from a cell expressing a polynucleotide selected from the group consisting of SEQ ID NO: 69, 73, 97, 99, 101, and 115.

Also provided are host cells comprising the polynucleotides encoding the antigen binding proteins of the present invention and methods of expressing the antigen binding proteins form said host cells. In addition, methods are provided for making the antigen binging proteins of the present invention.

Methods of making vectors, host cells and antibodies of the present invention include using conventional expression vectors or recombinant plasmids produced by placing coding sequences for the antibody in operative association with conventional regulatory control sequences capable of controlling the replication and expression in, and/or secretion from, a host cell. Regulatory sequences include promoter sequences, e.g., CMV promoter, and signal sequences, which can be derived from other known antibodies. Similarly, a second expression vector can be produced having a DNA sequence which encodes a complementary antibody light or heavy chain. Preferably this second expression vector is identical to the first except insofar as the coding sequences and selectable markers are concerned, so to ensure as far as possible that each polypeptide chain is functionally expressed. Alternatively, the heavy and light chain coding sequences for the altered antibody may reside on a single vector.

A selected host cell is co-transfected by conventional techniques with both the first and second vectors (or simply transfected by a single vector) to create the transfected host cell of the invention comprising both the recombinant or synthetic light and heavy chains. The transfected cell is then cultured by conventional techniques to produce the engineered antibody of the invention. The antibody which includes the association of both the recombinant heavy chain and/or light chain is screened from culture by appropriate assay, such as ELISA or RIA. Similar conventional techniques may be employed to construct other altered antibodies and molecules.

Suitable vectors for the cloning and subcloning steps employed in the methods and construction of the compositions of this invention may be selected by one of skill in the art. For example, the conventional pUC series of cloning vectors may be used. One vector, pUC19, is commercially available from supply houses, such as Amersham (Buckinghamshire, United Kingdom) or Pharmacia (Uppsala, Sweden). Additionally, any vector which is capable of replicating readily, has an abundance of cloning sites and selectable genes (e.g., antibiotic resistance), and is easily manipulated may be used for cloning. Thus, the selection of the cloning vector is not a limiting factor in this invention.

Similarly, the vectors employed for expression of the antibodies may be selected by one of skill in the art from any conventional vector. The vectors also contain selected regulatory sequences (such as CMV or RSV promoters) which direct the replication and expression of heterologous DNA sequences in selected host cells. These vectors contain the above described DNA sequences which code for the antibody or altered immunoglobulin coding region. In addition, the vectors may incorporate the selected immunoglobulin sequences modified by the insertion of desirable restriction sites for ready manipulation.

The expression vectors may also be characterized by genes suitable for amplifying expression of the heterologous DNA sequences, e.g., the mammalian dihydrofolate reductase gene (DHFR). Other preferable vector sequences include a poly A signal sequence, such as from bovine growth hormone (BGH) and the betaglobin promoter sequence (betaglopro). The expression vectors useful herein may be synthesized by techniques well known to those skilled in this art.

The components of such vectors, e.g. replicons, selection genes, enhancers, promoters, signal sequences and the like, may be obtained from commercial or natural sources or synthesized by known procedures for use in directing the expression and/or secretion of the product of the recombinant DNA in a selected host. Other appropriate expression vectors of which numerous types are known in the art for mammalian, bacterial, insect, yeast, and fungal expression may also be selected for this purpose.

The present invention also encompasses a cell line transfected with a recombinant plasmid containing the coding sequences of the antibodies or altered immunoglobulin molecules thereof. Host cells useful for the cloning and other manipulations of these cloning vectors are also conventional. However, most desirably, cells from various strains of E. coli are used for replication of the cloning vectors and other steps in the construction of altered antibodies of this invention.

Suitable host cells or cell lines for the expression of the antibody of the invention are preferably mammalian cells such as NSO, Sp2/0, CHO (e.g. DG44), COS, a fibroblast cell (e.g., 3T3), and myeloma cells, and more preferably a CHO or a myeloma cell. Human cells may be used, thus enabling the molecule to be modified with human glycosylation patterns. Alternatively, other eukaryotic cell lines may be employed. The selection of suitable mammalian host cells and methods for transformation, culture, amplification, screening and product production and purification are known in the art. See, e.g., Sambrook et al., cited above.

Bacterial cells may prove useful as host cells suitable for the expression of the recombinant Fabs of the present invention (see, e.g., Plückthun, A., Immunol. Rev., 130:151-188 (1992)). However, due to the tendency of proteins expressed in bacterial cells to be in an unfolded or improperly folded form or in a non-glycosylated form, any recombinant Fab produced in a bacterial cell would have to be screened for retention of antigen binding ability. If the molecule expressed by the bacterial cell was produced in a properly folded form, that bacterial cell would be a desirable host. For example, various strains of E. coli used for expression are well-known as host cells in the field of biotechnology. Various strains of B. subtilis, Streptomyces, other bacilli and the like may also be employed in this method.

Where desired, strains of yeast cells known to those skilled in the art are also available as host cells, as well as insect cells, e.g. Drosophila and Lepidoptera and viral expression systems. See, e.g. Miller et al., Genetic Engineering, 8:277-298, Plenum Press (1986) and references cited therein.

The general methods by which the vectors may be constructed, the transfection methods required to produce the host cells of the invention, and culture methods necessary to produce the antibody of the invention from such host cell are all conventional techniques. Typically, the culture method of the present invention is a serum-free culture method, usually by culturing cells serum-free in suspension. Likewise, once produced, the antibodies of the invention may be purified from the cell culture contents according to standard procedures of the art, including ammonium sulfate precipitation, affinity columns, column chromatography, gel electrophoresis and the like. Such techniques are within the skill of the art and do not limit this invention. For example, preparation of altered antibodies are described in WO 99/58679 and WO 96/16990.

Yet another method of expression of the antibodies may utilize expression in a transgenic animal, such as described in U.S. Pat. No. 4,873,316. This relates to an expression system using the animal's casein promoter which when transgenically incorporated into a mammal permits the female to produce the desired recombinant protein in its milk.

In a further aspect of the invention there is provided a method of producing an antibody of the invention which method comprises the step of culturing a host cell transformed or transfected with a vector encoding the light and/or heavy chain of the antibody of the invention and recovering the antibody thereby produced.

In one aspect, the present invention provides a method of inhibiting ADAM and ADAMTS activity by providing a molecule that simultaneously binds both catalytic and disintegrin domains. In certain embodiments the ADAMTS is ADAMTS 4 or ADAMTS5. In silico structure ‘best fit’ computational modeling using separate crystal structures for ADAMTS5 and the ADAMTS5 mAbs 12F4.1H7 and 7B4.1E11 suggest simultaneous antibody/antigen interactions between both the catalytic and disintegrin domains of ADAMTS5. The catalytic and disintegrin domains of ADAM and ADAMTS proteases are separated by a hinge region that imparts flexibility between the domains which may act to regulate function or allow for substrate localization to the catalytic site. The high affinity mAb binding observed at this domain spanning epitope likely ‘locks’ the catalytic and disintegrin domains of ADAMTS5 together thereby neutralizing enzymatic activity. In one embodiment, the molecule is an antibody that binds to both the disintegrin and catalytic domains simultaneously. In another embodiment the molecule is an antibody or antibody fragment of the present invention. In another embodiment, the present invention concerns an antibody which neutralizes the enzymatic activity of AMAMTS5, and in which the antibody simultaneously binds to catalytic and disintegrin domains with a KD of less than about 1×10⁻⁹ or 2×10⁻¹⁰ as measured by BiaCore or Octet QK.

In another embodiment of the present invention, pharmaceutical compositions are provided comprising at least one of the antigen binding proteins described herein. The current invention also provides use of at least one antigen binding protein to ADAMTS5 in the manufacture of a medicament for reducing at least one ADAMTS5 activity in a human. The present invention provides use of at least one antigen binding protein to ADAMTS5 for reducing at least one activity of ADAMTS5 in a human comprising administering to a patient in need thereof a composition comprising at least one antigen binding protein to ADAMTS5.

Pharmaceutical compositions of the present invention may further comprises a second antigen binding protein. In some instance the second antigen binding protein may be a monoclonal antibody. In one embodiment, the second monoclonal antibody binds at least one antigen selected from the group of ADAMTS4, ADAMTS5, NGF, OSM, TNF-α, IL-6, VIP, TRPV1, TRPV4, ADAMTS1, Aggrecan, Collagen II, RANKL, and/or IL-1. By way of example, the pharmaceutical compositions of the present invention may comprise a first antigen binding protein, which may be a monoclonal antibody to ADAMTS5 and a second monoclonal antibody, which may also bind ADAMTS5. By way of another example, a pharmaceutical composition of the present invention may comprise a first antigen binding protein, which is a monoclonal antibody that binds to ADAMTS5 and a second antigen binding protein, which is a monoclonal antibody that binds one of the following: ADAMTS4, NGF, OSM, TNF-α, IL-6, VIP, TRPV1, TRPV4, ADAMTS1, Aggrecan, Collagen II, RANKL, and/or IL-1.

Also provided are methods of treating a patient in need thereof comprising administering at least one dose of pharmaceutical composition of the present invention. In some aspects, the patient is suffering from a disease of the cartilage. A patient may be suffering from one or more diseases chosen from the group of: cancer, pain, chronic pain, neuropathic pain, postoperative pain, osteoarthritis, sports injuries, erosive arthritis, rheumatoid arthritis, psoriatic arthritis, Lyme arthritis, juvenile arthritis, ankylosing spondylosis, neuralgia, neuropathies, algesia, nerve injury, ischaemia, neurodegeneration, inflammatory diseases, cartilage degeneration, diseases affecting the larynx, trachea, auditory canal, intervertebral discs, ligaments, tendons, joint capsules or bone development, invertebral disc degeneration, osteopenia, or periodontal diseases, acute joint injury, and/or a disease related to joint destruction. In some instances, the patient is suffering from osteoarthritis.

In another embodiment, administering at least one dose of said pharmaceutical composition reduces cartilage degradation in said patient. In another embodiment, administering at least one dose of said pharmaceutical composition inhibits and/or reduces aggrecan cleavage in said patient.

Also provided herein are pharmaceutical compositions capable of treating disease associated with cartilage degradation or alleviating the symptoms produced thereby and formulated for the methods and uses described herein. The present invention provides an ADAMTS5 antibody for use in the treatment of diseases of the cartilage, for administration alone or in combination with at least one other therapeutic, including, but not limited to, at least one steroid and/or analgesic. Antigen-binding proteins of the present invention can be co-administered with other therapeutics in the same dose or separately. The present invention also provides ADAMTS5 antibodies or fragments thereof for all of the methods and uses described herein.

As used herein, “patient” refers to a human or other animal.

As used herein, “treatment” means: (1) the amelioration or prevention of the condition being treated or one or more of the biological manifestations of the condition being treated, (2) the interference with (a) one or more points in the biological cascade that leads to or is responsible for the condition being treated or (b) one or more of the biological manifestations of the condition being treated, or (3) the alleviation of one or more of the symptoms or effects associated with the condition being treated. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “safe and effective amount” means an amount of at least one antigen binding protein sufficient to significantly induce a positive modification in the condition to be treated but low enough to avoid serious side effects (at a reasonable benefit/risk ratio) within the scope of sound medical judgment. A safe and effective amount of at least one antigen binding protein of the invention will vary with the particular compound chosen (e.g. consider the potency, efficacy, and half-life of the compound); the route of administration chosen; the condition being treated; the severity of the condition being treated; the age, size, weight, and physical condition of the patient being treated; the medical history of the patient to be treated; the duration of the treatment; the nature of concurrent therapy; the desired therapeutic effect; and like factors, but can nevertheless be routinely determined by the skilled artisan.

The antigen binding proteins of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion, including intraarticular administration. Inhalation refers to administration into the patient's lungs whether inhaled through the mouth or through the nasal passages. Topical administration includes application to the skin as well as intraocular, otic, intravaginal, and intranasal administration.

The antigen binding proteins of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a antigen binding protein of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

In certain embodiments the antibody is used to deliver a drug to the cartilage. Such a drug could be an aggrecanase inhibitor, an anti-inflammatory drug, steroid or a drug related to pain management. Accordingly, in one aspect the invention is a method of delivering a drug to cartilage comprising lining the drug to an antibody of the present invention. Such delivery can be conducted in vitro, ex vivo, or in vivo.

In another embodiment the antibody is used to deliver a growth factor to the cartilage which would promote the growth of new cartilage. Such growth factors include Bone Morphogenic proteins, particularly BMP-7. Such delivery can be conducted in vitro, ex vivo, or in vivo.

### EXAMPLES

The following examples illustrate various aspects of this invention.

### Example 1

### Production of Mabs to Human ADAMTS4 and Human ADAMTS5

Human ADAMTS5 and ADAMTS4 proteins were produced in transfected CHO cells and/or BacMam transduced HEK293 cells and isolated by conventional chromatography methods.

SJL mice were co-immunized with purified ADAMTS4 (full length) and ADAMTS5 (truncated, full length, Cat, Cat/dis domains). Immunogenicity was tested on sera from serial bleeds.

Splenocytes and lymph nodes were isolated and fused to mouse myeloma cells using a P3X63/Ag8.653-derived fusion partner. Immortalized antibody producing cells were generated. HAT selection was used to deselect unfused myeloma cells.

Resulting hybridoma supernatants from active cultures were screened for specific binding and neutralization of recombinant human ADAMTS5 and ADAMTS4. Hits were identified, confirmed and cloned to monoclonality either by limiting dilution or growth in semi-solid media.

Monoclonal antibodies with desired characteristics were scaled up in liquid culture and the antibody was purified by standard chromatography methods. Resulting purified antibody clones were then further characterized for binding affinity and functional potency.

### Example 2

### Murine Antibodies Characterization

ADAMTS5 mAbs were characterized for neutralization potency using in vitro aggrecan substrate cleavage assays (Table 1). ADAMTS5 mAbs were characterized for affinity using both Octet QK (Table 1) and BiaCore (comparable, but not shown) technologies. Antibodies were also tested for cross-reactivity to Human ADAMTS1, ADAMTS4, ADAMTS13, MMP1, MMP3, MMP9 and MMP13 by celTRF and Octet QK, all of which were negative (not shown). All mAbs were also assessed for orthologue cross-reactivity by binding and neutralization against mouse, canine, and cynomolgus monkey ADAMTS5 (Table 1). Binding was also detected against rat ADAMTS5 (not shown). Affinity comparisons for murine and chimeric forms of anti-human ADAMTS5 mAbs on Octet QK are summarized in Table 2.

TABLE 1
Characterization of purified anti-human ADAMTS5 monoclonal antibodies.

| | | | | Aggrecan | | | |
|---|---|---|---|---|---|---|---|
| | | | | Substrate | | | |
| | | | | Cleavage | ADAMTS5 Orthologue | | |
| | Affinity (Qctet QK) | | | Neutralization | (Binding/Neutralization) | | |
| mAb | Ka | Kd | KD | IC50 (nM) | Mouse | Cynomolgus | Canine |
| 1G10.1C9 | 4.47E+04 | 8.43E−06 | 1.88E−10 | 0.375 | (+/+) | (+/+) | (+/+) |
| 2D3.1D4 | 6.67E+04 | 5.75E−06 | 8.62E−11 | 0.031 | (+/+) | (+/+) | (+/+) |
| 3A12.1D7 | 5.67E+04 | 4.67E−06 | 8.24E−11 | 0.769 | (+/+) | (+/+) | (+/+) |
| 5F10.1H6 | 5.53E+04 | 6.99E−06 | 1.26E−10 | 0.05 | (+/+) | (+/+) | (+/+) |
| 11F12.1D12 | 6.57E+04 | 4.33E−05 | 6.58E−10 | 2.527 | (+/+) | (+/+) | (+/+) |
| 12F4.1H7 | 3.31E+04 | 1.44E−05 | 4.36E−10 | 0.06 | (+/+) | (+/+) | (+/+) |
| 7B4.1E11 | 4.86E+04 | 4.36E−05 | 8.99E−10 | 0.08 | (+/+) | (+/+) | (+/+) |

TABLE 2
Direct comparison of purified murine and chimeric anti-human
ADAMTS5 mAbs

| | Affinity (Qctet QK) | | |
|---|---|---|---|
| mAb | Ka | Kd | KD |
| Murine 12F4.1H7 | 5.64E+04 | 3.86E−06 | 6.85E−11 |
| Chimeric 12F4.1H7 | 5.55E+04 | 4.43E−06 | 7.99E−11 |
| Murine 7B4.1E11 | 7.65E+04 | 2.74E−05 | 3.58E−10 |
| Chimeric 7B4.1E11 | 7.18E+04 | 2.47E−05 | 3.45E−10 |

### Example 3

### Sequences

Based on the characteristics identified in Example 2, six monoclonal antibodies were identified. The variable regions of these antibodies were sequenced. Alignments are shown below (Table 3). A consensus (majority) heavy chain variable region and light chain variable region are represented by SEQ ID NOs: 30 and 31 below. Heavy chain variable regions for mAb designates 12F4.1H7, 1G10.1C9, 2D3.1D4, 3A12.1D7, 5F10.1H6, and 7B4.1E11 are represented by SEQ ID NOs: 32-37, respectively, and encoded by SEQ ID NOs: 147, 157, 151, 153, 155, and 149, respectively. Light chain variable regions for mAb designates 2D3.1D4, 3A12.1D7, 5F10.1H6, 7B4.1E11 and 12F4.1H7, and are represented by SEQ ID NOs: 38-42, respectively and encoded by SEQ ID NOs: 152, 154, 156, 150, and 148, respectively

TABLE 3
anti-ADAMTS5 mAB CDR sequence alignment

V_{H} CDR Alignments

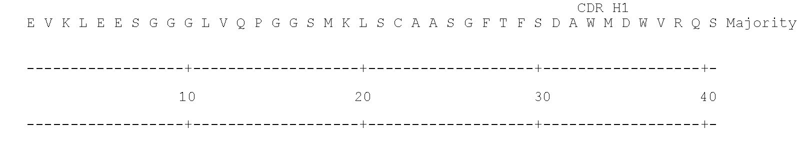

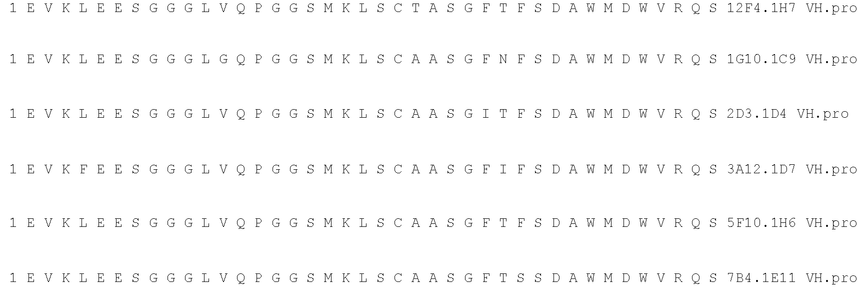

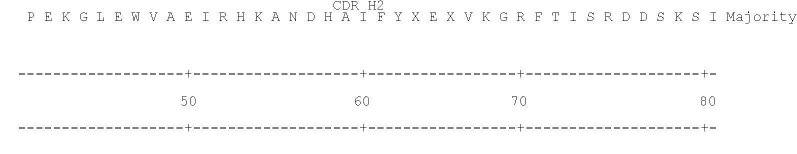

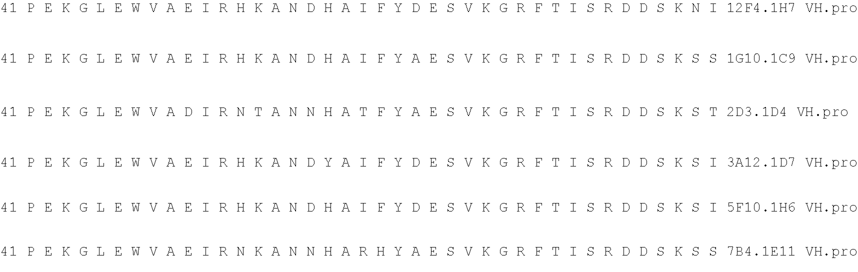

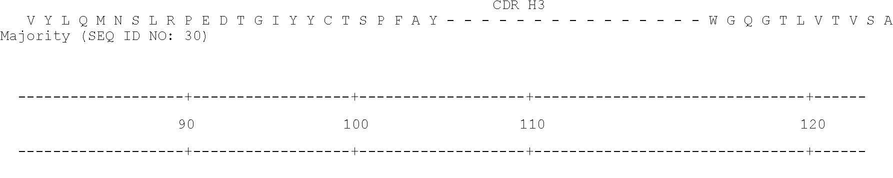

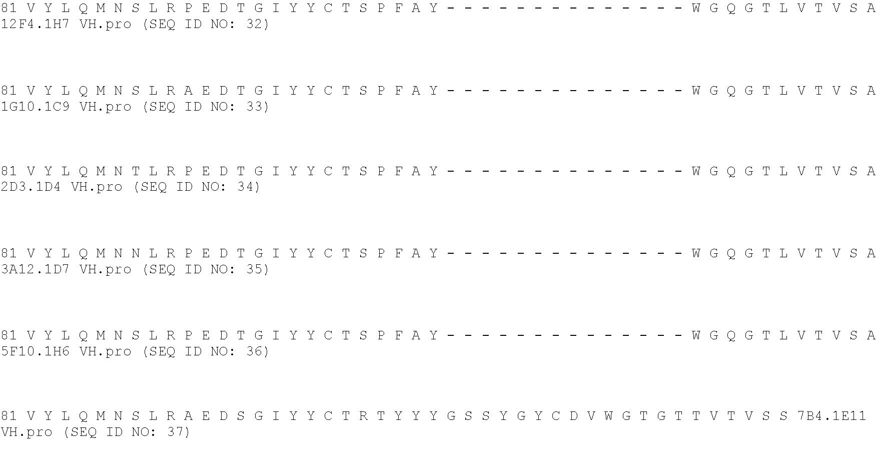

V_{L} Alignments

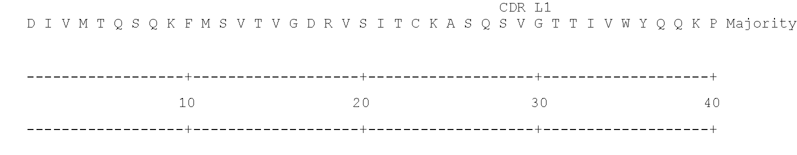

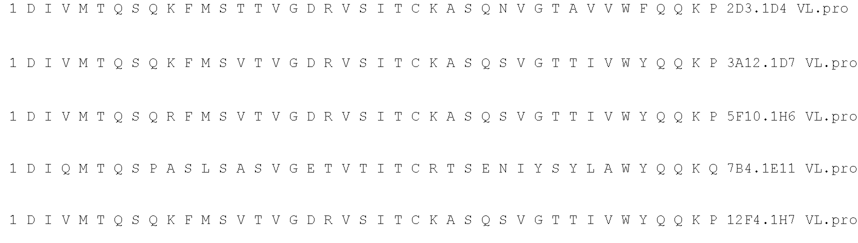

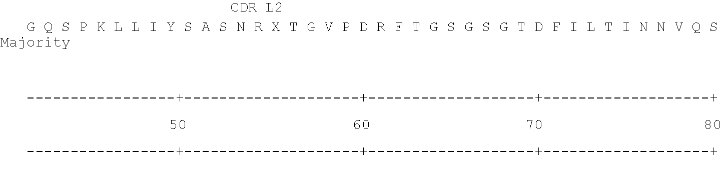

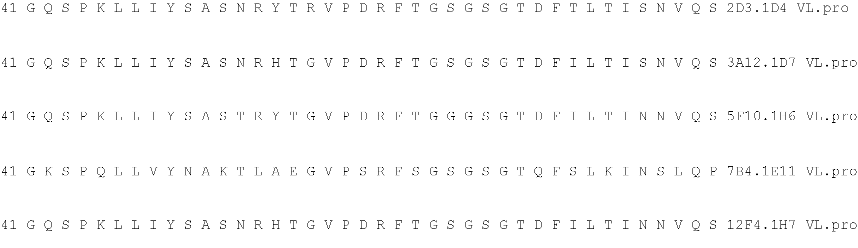

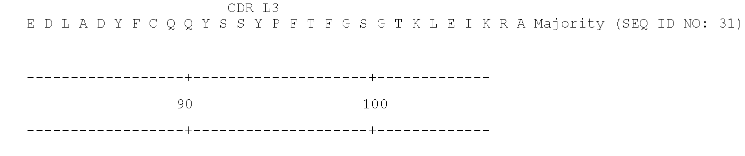

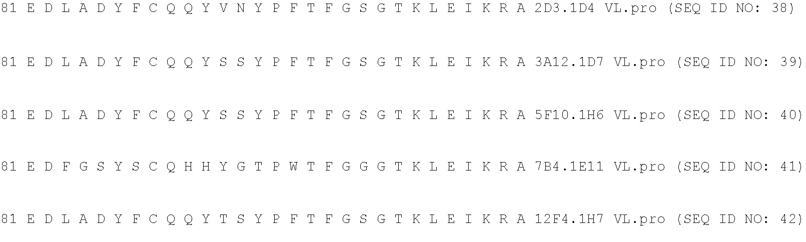

### Example 4

### Human OA Cartilage Explant

Donor human OA cartilage was obtained from knee replacement surgeries. Cartilage was processed from the bone and cut into 3 mm diameter discs. Discs were randomized and cultured in 96-well plates. Endogenous disease factors in the tissues were allowed to progress for cartilage degradation ex vivo. Samples were treated with the following: matched control IgG isotype, select anti-ADAMTS 5 antibodies (designated as 7B4.1E11 and 12F4.1H7), a selected anti-ADAMTS 4 antibody (designated as 7E8.1E3), or a known aggrecanase/MMP inhibitor, shown as GSK571949 (CAS number 329040-94-0) below. Each treatment condition was tested in multiples of 8 on each donor plate. Inhibition of ARGSVIL (SEQ ID NO:1) neoepitope release was measured for each sample at numerous points throughout the course of the experiment.

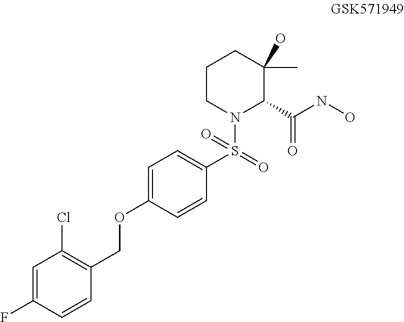

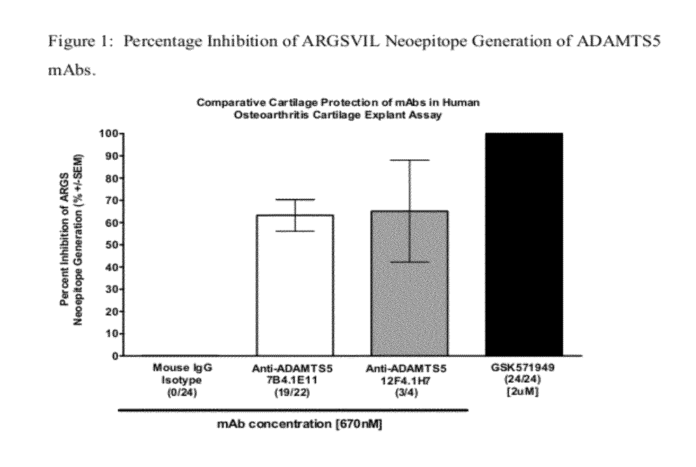

As described previously, cleavage of aggrecan by aggrecanase typically occurs at a conserved region within the interglobular domain of aggrecan. Enzyme cleavage will produce a released fragment containing a neoepitope with an N-terminal amino acid sequence (ARGSVIL) from aggrecan. This cleavage neoepitope can be detected and quantified using a monoclonal antibody which specifically binds to the cleaved forms, but not intact aggrecan. Both ADAMTS5 antibodies and the ADAMTS5 inhibitor showed significantly greater inhibition of ARGSVIL release than the control and ADAMTS4 antibody. A summary of percent inhibition of ARGSVIL release is shown in FIG. 1. The results demonstrate that ADAMTS5 specific mAbs are able to inhibit degradation at a rate of approximately 70% for the 2-3 week assessment period as compared to a small molecule assay control compound. These results are consistent within and across many individual donor samples.

### Example 5

### ADAMTS5 Antibody Dose Response in Human OA Cartilage Explant

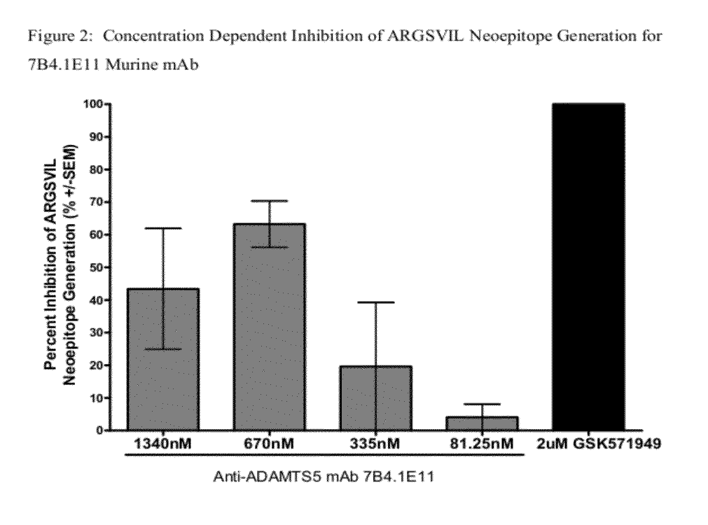

Percent inhibition of ARGSVIL release was tested as described in Example 4 for a dose response of a selected anti-ADAMTS5 antibody (see FIG. 2). Murine antibody 7B4.1E11 was tested at the following doses: 1340 nM, 670 nM, 335, nM, 81.25 nM. Control aggrecanase/MMP inhibitor was also used. Percent ARGSVIL release was lowest with the highest doses of anti-ADAMTS5 and declined upon treatment with lower doses. Matched isotype control antibody treatment doses (not shown) were used to determine 0% inhibition and a single effective dose of GSK571949 was used to calculate 100% inhibition. This mAb treatment demonstrates a dose response that appears to reach a maximal effect at the 670 nM dose (FIG. 2). However, it should be noted that more donors (n=22) were represented in the 670 nM dose point, while the other doses include fewer (n≦5).

### Example 6

### In Vivo Studies DMM

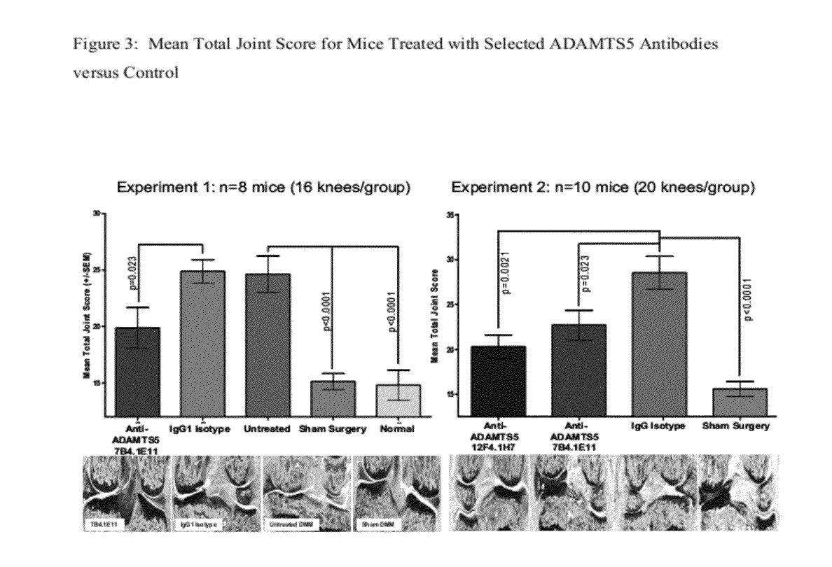

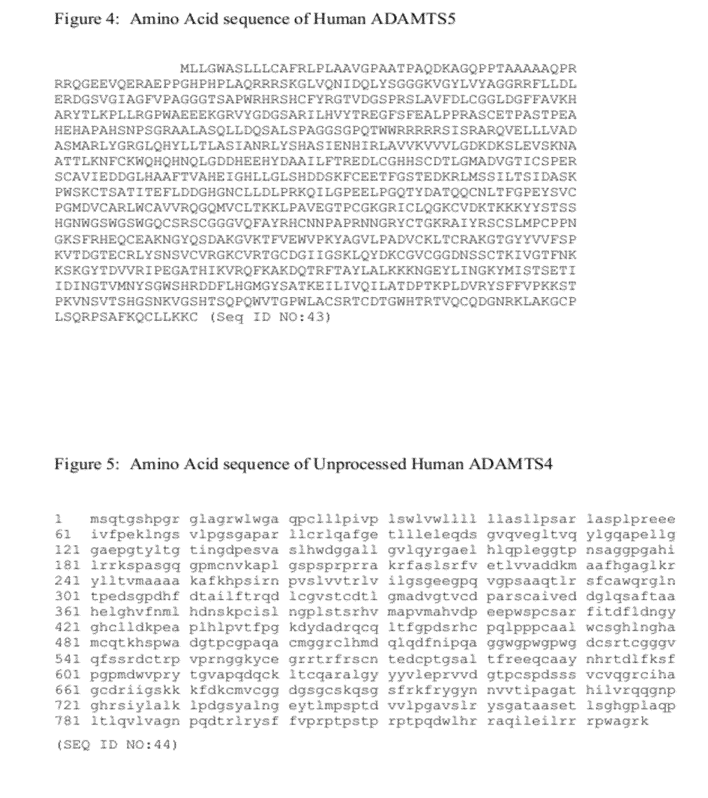

OA was induced in mice using a Destabilization Medial Meniscus (DMM) model for assessment of anti-ADAMTS5 antibody efficacy (see FIG. 3). Two separate experiments are shown. Three days prior to surgical DMM, mice were administered a 0.5 mg/dose of one of the following antibodies: anti-ADAMTS5 (7B4.1E11), anti-ADAMTS5 (12F4.1H7), or IgG isotype. Control groups included untreated mice with DMM surgery, mice with sham surgery and normal mice. Six days after dosing, mice were sacrificed and histopathology was performed by blinded investigators from which a joint score was given to each mouse knee assessed. Anti-ADAMTS5 antibodies showed significantly better mean joint scores compared with IgG1 isotype control. Additionally, sham surgery knees and normal knees had significantly better mean joint scores compared with untreated knees and IgG isotype.

### Example 7

### Monoclonal Antibodies Penetrate Cartilage in Vitro and in Vivo

The ability of therapeutic antibodies to penetrate tissue and reach the site of disease and their specific target is critical for efficacy. Aggrecanases, although classified as secreted proteins, have been shown to preferentially localize to the pericellular regions of chondrocytes within the cartilage matrix. Therefore, in order for a therapeutic mAb to reach an aggrecanase target it may require penetration through the cartilage matrix.

Initially, assessing the ability of a mAb to penetrate human cartilage was performed on ex-vivo tissue using mAbs with multiple specificities, including selected anti-ADAMTS5 mAbs. Full thickness cartilage plugs, spanning synovial surface through sub-chondral bone, from knee replacement surgical specimen were placed in tissue culture for defined durations in the presence of mouse monoclonal antibodies with specificities for human proteins located on the surface of chondrocytes or non-specific isotype controls. At the end of each timepoint tissues were processed for full thickness assessment, sectioned and stained using a FITC-labeled anti-mouse detection antibody. Penetration is defined by the depth and intensity of chondrocyte staining within the cartilage tissue. Irrespective of target specificity, mAb penetration was observed to be a concentration and time-dependent process primarily originating from the synovial surface of the cartilage and proceeding to full thickness penetration within 3-4 days dependent on concentration (not shown). No staining was observed for cartilage plugs treated with isotype control mAbs (not shown).

In vivo assessment of cartilage penetration was performed using near-infrared (NIR) dye labeled monoclonal antibodies, including selective mAbs for ADAMTS4, ADAMTS5 and isotype controls. NIR labeled mAbs were systemically (intraperitoneally) administered (0.5 mg dose) to mice who, six weeks earlier, had undergone surgical induction of osteoarthritis (DMM). Biodistribution of mAbs was monitored in the whole animal at numerous timepoints following administration using a Licor Odyssey system. Four days after mAb administration mice were sacrificed and imaging of the knee joint was performed on the Odyssey. Knee joints were then processed and sectioned for high resolution analysis on a microscope equipped with filters and a camera for NIR detection. Within four days after systemic mAb administration full thickness cartilage penetration could be observed for an anti-ADAMTS5 and anti-ADAMTS4 mAb, while no specific staining was observed for the isotype control mAb (not shown). Characteristic pericellular chondrocyte staining patterns were observed for ADAMTS5 and ADAMTS4 specific mAbs (not shown).

### Example 8

### Antibody Humanization—Cloning of Hybridoma Variable Regions

Total RNA was extracted from 7B4.1E11 and 12F4.1 H7 hybridoma cells, heavy and light variable domain cDNA sequence was then generated by reverse transcription and polymerase chain reaction (RT-PCR). The forward primer for RT-PCR was a mixture of degenerate primers specific for murine immunoglobulin gene leader-sequences and the reverse primer was specific for the antibody constant regions, in this case isotype IgG1 for 7B4.1E11 and IgG2 for 12F4.1H7. Primers were designed based on a strategy described by Jones and Bendig (Bio/Technology 9:88, 1991). RT-PCR was carried out for both V-region sequences to enable subsequent verification of the correct V-region sequences. The V-region products generated by the RT-PCR were cloned (Invitrogen TA Cloning Kit) and sequence data obtained.

### Example 9

### Antibody Humanization—Cloning of the chimera

The DNA expression constructs encoding the chimeric antibody were prepared de novo by build-up of overlapping oligonucleotides including restriction sites for cloning into mammalian expression vectors as well as a human signal sequence. HindIII and SpeI restriction sites were introduced to frame the V_{H} domain containing the signal sequence (SEQ ID NO: 45) for cloning into mammalian expression vectors containing the human γ1 constant region. HindIII and BsiWI restriction sites were introduced to frame the V_{L} domain containing the signal sequence (SEQ ID NO: 45) for cloning into mammalian expression vector containing the human kappa constant region.

### Example 10

### Antibody Humanization—Cloning of the Humanized Variants

The DNA expression constructs encoding the humanized antibody variants were prepared de novo by build-up of overlapping oligonucleotides including restriction sites for cloning into mammalian expression vectors as well as a human signal sequence. HindIII and SpeI restriction sites were introduced to frame the V_{H} domain containing the signal sequence (SEQ ID NO: 45) for cloning into mammalian expression vectors containing the human γ1 constant region. HindIII and BsiWI restriction sites were introduced to frame the V_{L} domain containing the signal sequence (SEQ ID NO: 45) for cloning into mammalian expression vector containing the human kappa constant region.

TABLE 4
Antibody variants

| | | | SEQ ID | SEQ ID |
|---|---|---|---|---|
| | | | NO: of | NO: of |
| Antibody | Alternative | | nucleotide | amino acid |
| ID | Names | Description | sequence | sequence |
| BPC1622 | 7B4 Chimera | 7B4 chimeric heavy | 67 | 68 |
| | | chain | | |
| | | 7B4 chimeric light | 69 | 70 |
| | | chain | | |
| BPC1623 | 12F4 Chimera | 12F4 chimeric heavy | 71 | 72 |
| | | chain | | |
| | | 12F4 chimeric light | 73 | 74 |
| | | chain | | |
| BPC1634 | 7B4 H0L0 | 7B4 H0 heavy chain | 83 | 84 |
| | | 7B4 L0 light chain | 97 | 98 |
| BPC1635 | 7B4 H1L0 | 7B4 H1 heavy chain | 85 | 86 |
| | | 7B4 L0 light chain | 97 | 98 |
| BPC1636 | 7B4 H2L0 | 7B4 H2 heavy chain | 87 | 88 |
| | | 7B4 L0 light chain | 97 | 98 |
| BPC1637 | 7B4 H3L0 | 7B4 H3 heavy chain | 89 | 90 |
| | | 7B4 L0 light chain | 97 | 98 |
| BPC1638 | 7B4 H4L0 | 7B4 H4 heavy chain | 91 | 92 |
| | | 7B4 L0 light chain | 97 | 98 |
| BPC1639 | 7B4 H5L0 | 7B4 H5 heavy chain | 93 | 94 |
| | | 7B4 L0 light chain | 97 | 98 |
| BPC1640 | 7B4 H6L0 | 7B4 H6 heavy chain | 95 | 96 |
| | | 7B4 L0 light chain | 97 | 98 |
| BPC1641 | 7B4 H0L1 | 7B4 H0 heavy chain | 83 | 84 |
| | | 7B4 L1 light chain | 99 | 100 |
| BPC1642 | 7B4 H1L1 | 7B4 H1 heavy chain | 85 | 86 |
| | | 7B4 L1 light chain | 99 | 100 |
| BPC1643 | 7B4 H2L1 | 7B4 H2 heavy chain | 87 | 88 |
| | | 7B4 L1 light chain | 99 | 100 |
| BPC1644 | 7B4 H3L1 | 7B4 H3 heavy chain | 89 | 90 |
| | | 7B4 L1 light chain | 99 | 100 |
| BPC1645 | 7B4 H4L1 | 7B4 H4 heavy chain | 91 | 92 |
| | | 7B4 L1 light chain | 99 | 100 |
| BPC1646 | 7B4 H5L1 | 7B4 H5 heavy chain | 93 | 94 |
| | | 7B4 L1 light chain | 99 | 100 |
| BPC1647 | 7B4 H6L1 | 7B4 H6 heavy chain | 95 | 96 |
| | | 7B4 L1 light chain | 99 | 100 |
| BPC1648 | 7B4 H0L2 | 7B4 H0 heavy chain | 83 | 84 |
| | | 7B4 L2 light chain | 101 | 102 |
| BPC1649 | 7B4 H1L2 | 7B4 H1 heavy chain | 85 | 86 |
| | | 7B4 L2 light chain | 101 | 102 |
| BPC1650 | 7B4 H2L2 | 7B4 H2 heavy chain | 87 | 88 |
| | | 7B4 L2 light chain | 101 | 102 |
| BPC1651 | 7B4 H3L2 | 7B4 H3 heavy chain | 89 | 90 |
| | | 7B4 L2 light chain | 101 | 102 |
| BPC1652 | 7B4 H4L2 | 7B4 H4 heavy chain | 91 | 92 |
| | | 7B4 L2 light chain | 101 | 102 |
| BPC1653 | 7B4 H5L2 | 7B4 H5 heavy chain | 93 | 94 |
| | | 7B4 L2 light chain | 101 | 102 |
| BPC1654 | 7B4 H6L2 | 7B4 H6 heavy chain | 95 | 96 |
| | | 7B4 L2 light chain | 101 | 102 |
| BPC1655 | 12F4 H0L0 | 12F4 H0 heavy chain | 103 | 104 |
| | | 12F4 L0 light chain | 113 | 114 |
| BPC1656 | 12F4 H1L0 | 12F4 H1 heavy chain | 105 | 106 |
| | | 12F4 L0 light chain | 113 | 114 |
| BPC1657 | 12F4 H2L0 | 12F4 H2 heavy chain | 107 | 108 |
| | | 12F4 L0 light chain | 113 | 114 |
| BPC1658 | 12F4 H3L0 | 12F4 H3 heavy chain | 109 | 110 |
| | | 12F4 L0 light chain | 113 | 114 |
| BPC1659 | 12F4 H4L0 | 12F4 H4 heavy chain | 111 | 112 |
| | | 12F4 L0 light chain | 113 | 114 |

### Example 11

### Antibody Humanization—Expression of the Recombinant Antibodies (Including Antibody Quantification)

Expression plasmids encoding the heavy and light chains respectively were transiently co-transfected into HEK 293 6E cells and expressed at small scale to produce antibody. Heavy and light chains of the 7B4 and 12F4 chimeric antibodies and an irrelevant control antibody were also expressed. Antibodies were quantified by ELISA. ELISA plates were coated with anti human IgG (Sigma I3382) at 1 μg/ml and blocked with blocking solution (4% BSA in Tris buffered saline). Various dilutions of the tissue culture supernatants were added and the plate was incubated for 1 hour at room temperature. Dilutions of a known standard antibody were also added to the plate. The plate was washed in TBST and binding was detected by the addition of a peroxidase labelled anti human kappa light chain antibody (Sigma A7164) at a dilution of 1/1000 in blocking solution. The plate was incubated for 1 hour at room temp before washing in TBST. The plate was developed by addition of OPD substrate (Sigma P9187) and colour development stopped by addition of 2M H₂SO₄. Absorbance was measured at 490 nm and a standard curve plotted using data for the known standard dilutions. The standard curve was used to estimate the concentration of antibody in the tissue culture supernatants.

### Example 12

### ADAMTS5 Binding ELISA

A binding ELISA was carried out to test the binding of the expressed antibodies in cell culture supernatant to recombinant ADAMTS5. ELISA plates were coated with recombinant human ADAMTS5 at 0.2 μg/ml and blocked with blocking solution (4% BSA in Tris buffered saline). Various dilutions of the tissue culture supernatants were added and the plate was incubated for 1 hour at room temperature before washing in TBST. Binding was detected by the addition of a peroxidase labelled anti human kappa light chain antibody (Sigma A7164) at a dilution of 1/1000 in blocking solution. The plate was incubated for 1 hour at room temp before washing in TBST. The plate was developed by addition of OPD substrate (Sigma P9187) and colour development stopped by addition of 2M H₂SO₄. Absorbance was measured at 490 nm with a plate reader and the mean absorbance plotted against concentration.

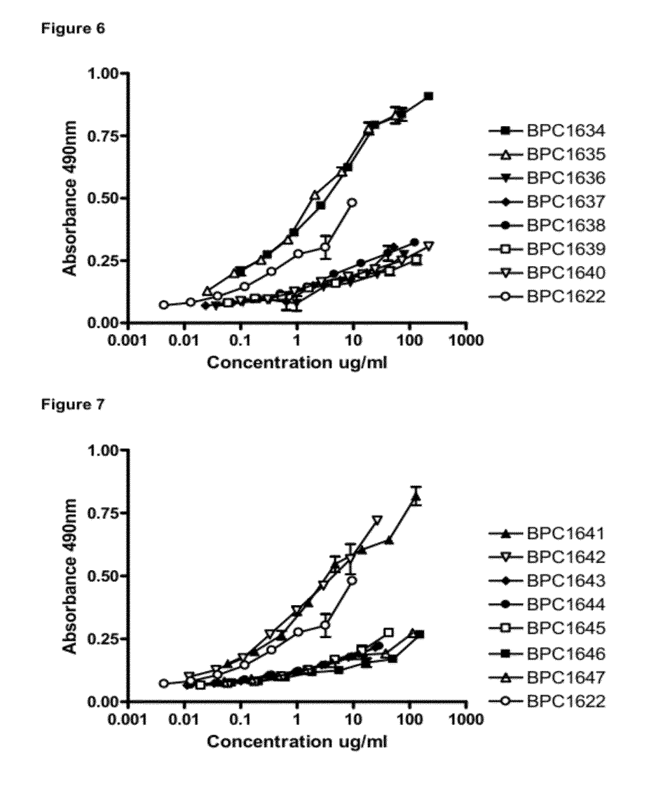

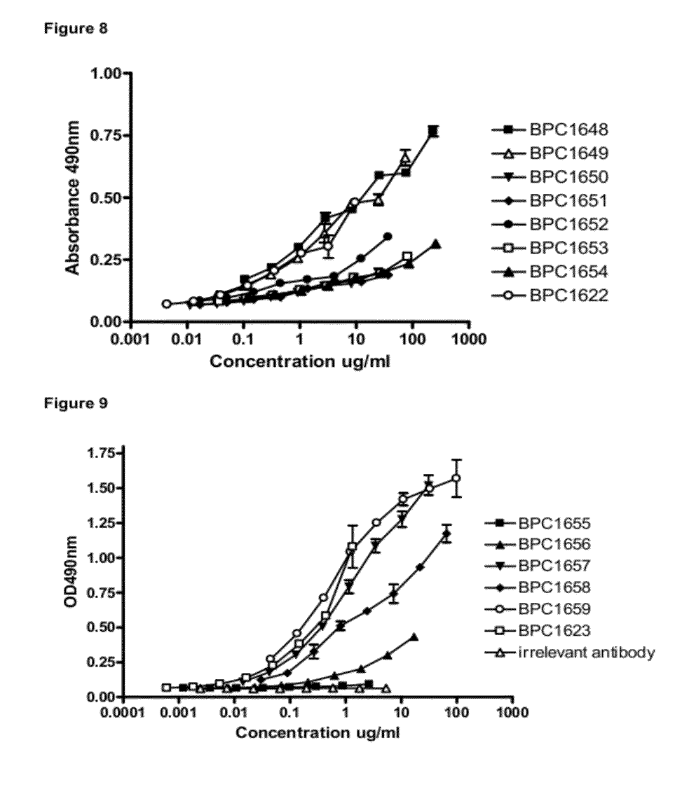

FIGS. 6-9 show the binding of the humanized anti ADAMTS5 antibodies to recombinant antigen.

### Example 13

### BIAcore

Anti-human IgG (Biacore™, BR-1008-39) was immobilized on a CM5 chip by primary amine coupling. This surface was then used to capture the humanized antibodies, ADAMTS5 (R&D Systems 2198-AD) was then passed over the captured antibody at a single concentration of 64 nM, regeneration was carried out using 100 mM phosphoric acid followed by 3M MgCl₂. The binding curves were double referenced with buffer injection (i.e. 0 nM) and the data was fitted to the T100 analysis software using the 1:1 model. The run was carried out at 25° C., using HBS-EP as the running buffer. The data obtained is shown in Table 5. All antibodies were captured from tissue culture supernatants unless specified.

TABLE 5
Kinetics of binding to human ADAMTS5

| Sample | ka (1/Ms) | kd (1/s) | KD (nM) |
|---|---|---|---|
| BPC1623 | 6.463E+05 | 4.700E−05 | 0.07273 |
| (purified) | | | |
| BPC1622 | 8.723E+05 | 1.056E−03 | 1.211 |
| (purified) | | | |
| BPC1622 | 1.159E+06 | 1.305E−03 | 1.125 |
| BPC1623 | 6.234E+05 | 4.823E−05 | 0.07737 |
| BPC1634 | 5.538E+05 | 4.811E−04 | 0.8687 |
| BPC1635 | 7.041E+05 | 8.258E−04 | 1.173 |
| BPC1636 | 1.073E+06 | 8.963E−04 | 0.8357 |
| BPC1637 | 1.119E+06 | 9.625E−04 | 0.8604 |
| BPC1638 | 8.749E+05 | 8.309E−04 | 0.9497 |
| BPC1639 | 9.271E+05 | 8.937E−04 | 0.9639 |
| BPC1640 | 7.870E+05 | 8.492E−04 | 1.079 |
| BPC1641 | 7.144E+05 | 9.453E−04 | 1.323 |
| BPC1642 | 8.874E+05 | 1.210E−03 | 1.364 |
| BPC1643 | 1.362E+06 | 1.434E−03 | 1.053 |
| BPC1644 | 1.491E+06 | 1.572E−03 | 1.054 |
| BPC1645 | 1.119E+06 | 1.159E−03 | 1.036 |
| BPC1646 | 1.788E+06 | 1.843E−03 | 1.031 |
| BPC1647 | 1.485E+06 | 1.453E−03 | 0.9784 |
| BPC1648 | 7.875E+05 | 9.512E−04 | 1.208 |
| BPC1649 | 1.023E+06 | 1.297E−03 | 1.267 |
| BPC1650 | 3.140E+06 | 3.427E−03 | 1.091 |
| BPC1651 | 2.653E+06 | 3.150E−03 | 1.188 |
| BPC1652 | 1.963E+06 | 2.122E−03 | 1.081 |
| BPC1653 | 2.236E+06 | 2.597E−03 | 1.161 |
| BPC1654 | 1.040E+06 | 1.137E−03 | 1.094 |
| BPC1655 | no | binding | seen |
| BPC1656 | 6.327E+05 | 3.950E−04 | 0.6243 |
| BPC1657 | 5.850E+05 | 7.540E−05 | 0.1289 |
| BPC1658 | 4.483E+05 | 1.398E−04 | 0.3119 |
| BPC1659 | 4.894E+05 | 4.147E−05 | 0.08475 |

### Example 14

### Comparative Affinity of Murine, Chimeric and Humanized ADAMTS5 mAbs

Affinity was assessed for the mAbs in two formats (antigen on sensor, Octet QK and antibody on sensor, Biacore) (Table 6). The relevance of the Octet QK format, which would be representative of a cellular target, is relevant to ADAMTS5 due to the naturally occurring cell associated form of the enzyme in vivo. However, since ADAMTS5 can also be found in a secreted form, we also assessed affinity in an antibody on sensor format using Biacore. Differences observed between the Octet and Biacore analysis may represent format variations (i.e. antigen on sensor in Octet QK and mAb on sensor in Biacore) and technical differences between the systems. However, when run in the same formats the systems were remarkably similar in terms of overall KD for these mAbs. Differences in Kd and Ka were observed, likely due to instrument design differences.

The humanized CS mAb (12F4 H4L0) exhibits an overall affinity (1(D) of 38.3 pM as measured in the antigen down format in the Octet QK system. The 12F4 H4L0 mAb demonstrates a KD of 85 pM, in the mAb down format in the Biacore system. 7B4 H0L0 shown for reference, exhibits a KD of 205 and 869 pM in the Octet QK and Biacore systems respectively. Affinity values for the murine and chimeric forms of each mAb are shown here for reference to demonstrate that affinity was retained post humanization. All mAbs in this experiment contained fully functional Fc portions (i.e., were not Fc-disabled).

TABLE 6
Comparative affinity of murine, chimeric and humanized ADAMTS5 mAbs

| | Affinity (Biacore) mAb Down no Avidity | | | Affinity (Octet QK) Antigen Down incl Avidity | | |
|---|---|---|---|---|---|---|
| mAb | Kₐ | K_{d} | KD | Kₐ | K_{d} | KD |
| Murine | 1.56E+06 | 1.04E−04 | 0.067 nM | 6.99E+04 | 2.49E−06 | 0.0356 nM |
| 12F4.1H7 | | | | | | |
| Chimeric | 1.56E+06 | 7.86E−05 | 0.051 nM | 6.85E+04 | 2.96E−06 | 0.0431 nM |
| 12F4.1H7 | | | | | | |
| Humanized | 4.89E+05 | 4.15E−05 | 0.085 nM | 7.54E+04 | 2.88E−06 | 0.0383 nM |
| 12F4.1H7 H4L0 | | | | | | |
| Murine | 4.81E+06 | 2.32E−03 | 0.483 nM | 9.25E+04 | 2.59E−05 | 0.280 nM |
| 7B4.1E11 | | | | | | |
| Chimeric | 2.43E+06 | 1.14E−03 | 0.473 nM | 1.00E+05 | 2.61E−05 | 0.260 nM |
| 7B4.1E11 | | | | | | |
| Humanized | 5.54E+05 | 4.81E−04 | 0.869 nM | 1.06E+05 | 2.16E−05 | 0.205 nM |
| 7B4.1E11 H0L0 | | | | | | |

### Example 15

### Human OA Cartilage Explant Experiment Humanized 12F4 H4L0 Dose Range

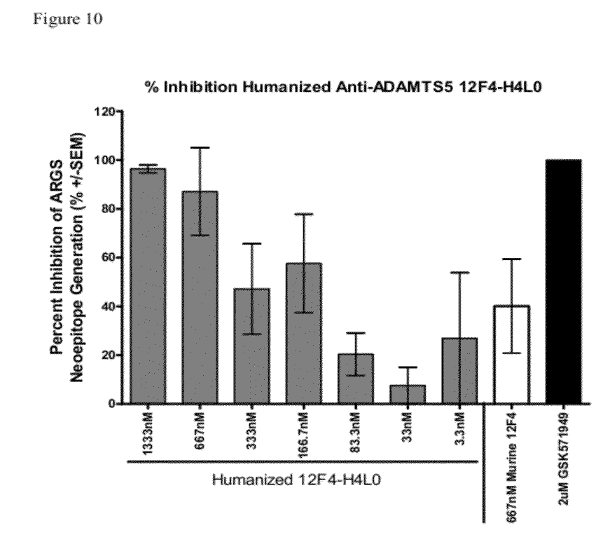

The humanized 12F4 H4L0 Fc disabled mAb was assessed in this system in a dose response format versus appropriate Fc disabled isotype and other positive and negative controls and in comparison to the parental murine 12F4.1H7 version at a single dose. Multiple donors (n=4) were assessed individually in this study and results were compiled to generate a mean inhibition score across an OA patient population. GSK571949 was used at a single concentration (2 uM) as an assay control to set maximal inhibition levels. The results demonstrate a clear 12F4 H4L0 dose response with a maximal response achieved at the highest dose (1333 nM) as compared to a dose range of irrelevant humanized isotype control mAb (FIG. 10). Nearly complete inhibition of ARGS neoepitope in relation to GSK571949 was achieved at the highest dose (2 uM). In addition, the level of inhibition for 12F4 H4L0 was demonstrated to be greater than 12F4.1H7 in these donors, suggesting retained efficacy following humanization. Although the level of efficacy is greater for the humanized version of the mAb it is not clear whether this is a true increase or due to differences in mAb preparations. It should be noted that the level of efficacy observed with the 12F4.1H7 form of the mAb in separate experiments with additional OA donors (FIG. 1) was greater than observed here. Taking into account the relevance of this system to the human disease and observed efficacy, this data strongly supports validation of this approach and these mAbs.

### Example 16

### Human OA Cartilage Explant Therapeutic Pulse Chase Experiment

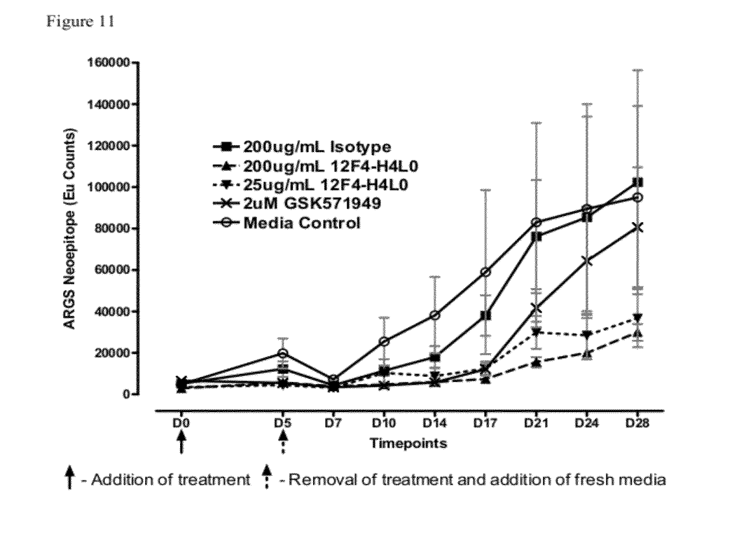

This experiment was designed as in Example 15, however following a 5 day treatment duration with the mAbs or compounds to allow for saturation of the system, the treatments were removed and replaced with fresh media lacking the treatment. The assay was continued for 4 weeks with periodic sampling of the culture media for subsequent assessment of cartilage degradation markers. Following each sampling, the same volume withdrawn in the sample was replaced with fresh media. This format was designed to address the potency and duration of the therapeutic effect and indirectly suggest ADAMTS5 turnover rate within the tissue. Results shown compiled from identical experiments using 4 different human OA donors (FIG. 11). These results provide evidence of the extended duration of the ADAMTS5 mAb response in human OA cartilage even in advanced disease state (i.e. at time of joint replacement). In addition, these results provide evidence of the low turnover rate of ADAMTS5 in diseased tissue.

### Example 17

### Fc-Disabled Humanized Antibody

The gene encoding the humanized 12F4 H4 V_{H} domain was cloned onto the modified human gamma 1 constant region, IgG1m(AA). The IgG1m(AA) constant region contains two alanine substitutions in the CH2 domain at positions L235 and G237(EU index numbering). These mutations render the resulting antibody unable to bind the necessary Fc receptors or complement component C1q, thus disabling its ability to induce antibody dependent cellular cytotoxicity (ADCC) or complement dependent cvtotoxi city (CDC).

TABLE 7

| | | | DNA | Amino Acid |
|---|---|---|---|---|
| | | | Sequence | Sequence |
| BPC1661 | 12F4 H4L0 | 12F4 H4 heavy chain | 159 | 158 |
| | IgG1m(AA) | 12F4 L0 light chain | 113 | 114 |

Expression plasmids encoding the heavy and light chains of the antibodies BPC1623 (12F4 Chimera), BPC1659 (12F4 H4L0 IgG1 wt) and BPC1661 (12F4 H4L0 IgG1m(AA)) were expressed in HEK cells. Antibodies were purified by Protein A affinity chromatography and quantified by spectrophotometry.

A binding ELISA was carried out to compare the binding of the purified antibodies to ADAMTS5. An irrelevant antibody of IgG1 wt isotype and an Fc disabled antibody were included as negative controls. Briefly, plates were coated with recombinant human ADAMTS5 at 0.2 ug/ml and blocked with blocking solution (4% BSA in Tris buffered saline). Various concentrations of purified antibody were added and the plate incubated for 1 hour at room temperature before washing with TBST (Tris buffered saline +0.05% Tween 20). Binding was detected by the addition of a peroxidase labelled anti human kappa light chain antibody (Sigma A7164) at a dilution of 1/1000 in blocking solution. The plate was incubated for 1 hour at room temperature before washing in TBST. The plate was developed by addition of OPD substrate (Sigma P9187) and colour development stopped by addition of 2M H₂SO₄. Absorbance was measured at 490 nm with a plate reader and the mean absorbance plotted against concentration.

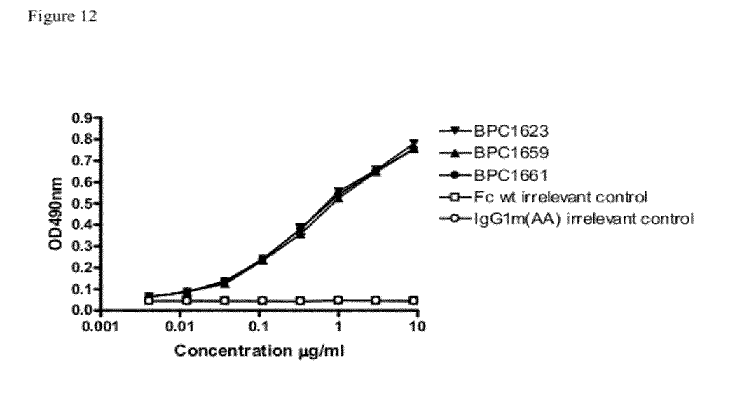

FIG. 12 shows the binding of the purified anti ADAMTS5 antibodies to recombinant antigen.

### Example 18

### Biacore of Fc-Disabled Antibody

Protein A was immobilized on a CM5 biosensor chip by primary amine coupling. This surface was used to capture the Fc disabled anti-ADAMTS5 antibody, 12F4 H4L0 IgG1m(AA) (BPC1661), (CHO and HEK expressed material). Recombinant human ADAMTS5 (R&D Systems 2198-AD) was used as the analyte at 64 nM, 16 nM, 4 nM, 1 nM, 0.25 nM and 0.0625 nM, with a buffer injection (i.e. 0 nM) used to double reference the binding curves. Regeneration of the capture surface (i.e. removal of the captured antibody) was with 50 mM NaOH. The running buffer was HBS-EP and the run was carried out on the Biacore T100 at 25° C. The data was fitted to the 1:1 model inherent to the machines analysis software. The results showed that there was no difference between material produced in different cell lines in terms of binding affinity.

Results

| Construct | ka | kd | KD (nM) |
|---|---|---|---|
| 12F4 H4L0 IgG1m(AA) (HEK | 9.43E+05 | 6.98E−05 | 0.074 |
| Expressed) | | | |
| 12F4 H4L0 IgG1m(AA) (CHO | 8.30E+05 | 5.85E−05 | 0.070 |
| Expressed) | | | |

### Example 19

### Crystallography Structure Modeling of Antigen-Antibody Interaction—Implications for Epitope and MOA

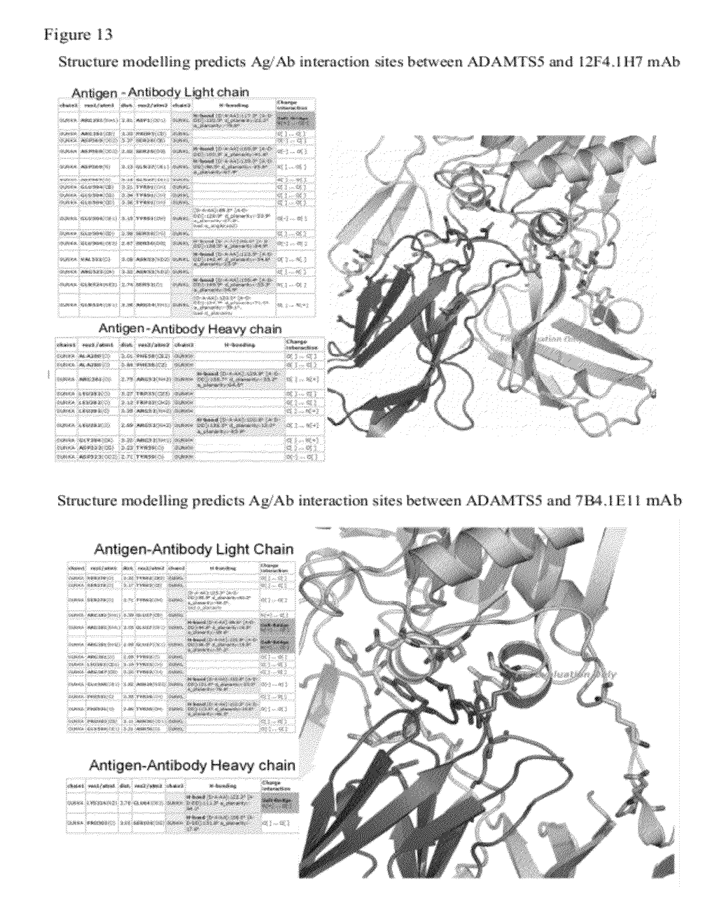

In silico structure ‘best fit’ computational modeling using separate crystal structures for ADAMTS5 and the ADAMTS5 mAb 12F4.1H7 suggest simultaneous antibody/antigen interactions between both the catalytic and disintegrin domains of ADAMTS5. The catalytic and disintegrin domains of ADAM and ADAMTS proteases are separated by a hinge region that imparts flexibility between the domains which may act to regulate function or allow for substrate localization to the catalytic site. The high affinity mAb binding observed at this domain spanning epitope likely ‘locks’ the catalytic and disintegrin domains of ADAMTS5 together thereby neutralizing enzymatic activity. Shown in FIG. 13 are the predicted amino acid sites of interaction between ADAMTS5 and the 12F4.1H7 and 7B4.1E11 mAbs.

| | Sequence identifier | |
|---|---|---|
| | (SEQ ID NO) | |
| | amino acid | DNA |
| Sequence Descriptions | sequence | sequence |
| Signal peptide sequence | 46 | 45 |
| 7B4 mouse variable heavy | 48 | 47 |
| 7B4 mouse variable light | 50 | 49 |
| 12F4 mouse variable heavy | 52 | 51 |
| 12F4 mouse variable light | 54 | 53 |
| 7B4 CDRH1 | 55 | |
| 7B4 CDRH2 | 56 | |
| 7B4 CDRH3 | 57 | |
| 7B4 CDRL1 | 58 | |
| 7B4 CDRL2 | 59 | |
| 7B4 CDRL3 | 60 | |
| 12F4 CDRH1 | 61 | |
| 12F4 CDRH2 | 62 | |
| 12F4 CDRH3 | 63 | |
| 12F4 CDRL1 | 64 | |
| 12F4 CDRL2 | 65 | |
| 12F4 CDRL3 | 66 | |
| 7B4 chimera heavy chain | 68 | 67 |
| 7B4 chimera light chain | 70 | 69 |
| 12F4 chimera heavy chain | 72 | 71 |
| 12F4 chimera light chain | 74 | 73 |
| 7B4 chimera heavy chain variable region | 76 | 75 |
| 7B4 chimera light chain variable region | 78 | 77 |
| 12F4 chimera heavy chain variable region | 80 | 79 |
| 12F4 chimera light chain variable region | 82 | 81 |
| 7B4 H0 heavy chain | 84 | 83 |
| 7B4 H1 heavy chain | 86 | 85 |
| 7B4 H2 heavy chain | 88 | 87 |
| 7B4 H3 heavy chain | 90 | 89 |
| 7B4 H4 heavy chain | 92 | 91 |
| 7B4 H5 heavy chain | 94 | 93 |
| 7B4 H6 heavy chain | 96 | 95 |
| 7B4 L0 light chain | 98 | 97 |
| 7B4 L1 light chain | 100 | 99 |
| 7B4 L2 light chain | 102 | 101 |
| 12F4 H0 heavy chain | 104 | 103 |
| 12F4 H1 heavy chain | 106 | 105 |
| 12F4 H2 heavy chain | 108 | 107 |
| 12F4 H3 heavy chain | 110 | 109 |
| 12F4 H4 heavy chain | 112 | 111 |
| 12F4 L0 light chain | 114 | 113 |
| 7B4 H0 heavy chain variable region | 116 | 115 |
| 7B4 H1 heavy chain variable region | 118 | 117 |
| 7B4 H2 heavy chain variable region | 120 | 119 |
| 7B4 H3 heavy chain variable region | 122 | 121 |
| 7B4 H4 heavy chain variable region | 124 | 123 |
| 7B4 H5 heavy chain variable region | 126 | 125 |
| 7B4 H6 heavy chain variable region | 128 | 127 |
| 7B4 L0 light chain variable region | 130 | 129 |
| 7B4 L1 light chain variable region | 132 | 131 |
| 7B4 L2 light chain variable region | 134 | 133 |
| 12F4 H0 heavy chain variable region | 136 | 135 |
| 12F4 H1 heavy chain variable region | 138 | 137 |
| 12F4 H2 heavy chain variable region | 140 | 139 |
| 12F4 H3 heavy chain variable region | 142 | 141 |
| 12F4 H4 heavy chain variable region | 144 | 143 |
| 12F4 L0 light chain variable region | 146 | 145 |
| 12F4.1H7 heavy chain variable region | 32 | 147 |
| 1G10.1C9 heavy chain variable region | 33 | 157 |
| 2D3.1D4 heavy chain variable region | 34 | 151 |
| 3A12.1D7 heavy chain variable region | 35 | 153 |
| 5F10.1H6 heavy chain variable region | 36 | 155 |
| 7B4.1E11 heavy chain variable region | 37 | 149 |
| 2D3.1D4 light chain variable region | 38 | 152 |
| 3A12.1D7 light chain variable region | 39 | 154 |
| 5F10.1H6 light chain variable region | 40 | 156 |
| 7B4.1E11 light chain variable region | 41 | 150 |
| 12F4.1H7 light chain variable region | 42 | 148 |
| 12F4 H4L0 IgG1m(AA) heavy chain | 158 | 159 |

1. An isolated antigen binding protein comprising at least one first immunoglobulin variable domain capable of binding to human ADAMTS5.

2. The antigen binding protein of claim 1, wherein said antigen binding protein is an antibody or fragment thereof.

3. The antigen binding protein of claim 1 or 2, wherein said antibody is a monoclonal antibody or fragment thereof.

4. The antigen binding protein of claim 3, wherein said monoclonal antibody or fragment thereof is mouse, chimeric, humanized, or fully human.

5. The antigen binding protein of any one of claims 1 to 4, wherein said antigen binding protein comprises at least one complementarity determining region.

6. The antigen binding protein of any one of claims 1 to 5, wherein said antigen binding protein is a monoclonal antibody comprising a heavy chain comprising CDRH1, CDRH2 and CDRH3 and a light chain comprising CDRL1, CDRL2 and CDRL3, wherein the complementarity determining regions (CDRs) of the heavy chain are selected from the group of:
a) CDRH1 having at least about 80% sequence identity to amino acid sequence DAWMD;
b) CDRH2 having at least about 70% sequence identity to amino acid sequence EIRHKANDHAIFYXESVKG; and
c) CDRH3 having at least about 70% sequence identity to amino acid sequence TYYYGSSYGYCDV or PFAY; and
the complementarity determining regions of the light chain are selected from the group of:
d) CDRL1 having at least about 70% sequence identity to amino acid sequence KASQSVGTTIV or RTSENIYSYLA;
e) CDRL2 having at least about 70% sequence identity to amino acid sequence NAKTLAE or SASNRXT; and
f) CDRL3 having at least about 70% sequence identity to amino acid sequence QQYSSYPFT or QHHYGTPWT.

7. The antigen binding protein of any one of claims 1 to 5, wherein said polypeptide is a monoclonal antibody comprising a heavy chain comprising CDRH1, CDRH2 and CDRH3 and a light chain comprising CDR1, CDRL2 and CDRL3, wherein the complementarity determining regions (CDRs) of the heavy chain are selected from:
(a) CDRH1 is amino acid sequence DAWMD;
(b) CDRH2 is select from amino acid sequence EIRHKANDHAIFYAESVKG, EIRNKANNHARHYAESVKG, EIRHKANDYAIFYDESVKG, EIRHKANDHAIFYDESVKG, or DIRNTANNHATFYAESVKG, EIRHKANDHAIFYDESVKG ; and
(c) CDRH3 is TYYYGSSYGYCDV or PFAY; and
the complementarity determining regions of the light chain are selected from:
(d) CDRL1 is select from amino acid sequence KASQSVGTTIV, RTSENIYSYLA, or KASQNVGTAVV;
(e) CDRL2 is select from amino acid sequence NAKTLAE, SASNRHT, SASTRYT, or SASNRYT; and
(f) CDRL3 is select from amino acid sequence QQYSSYPFT, QHHYGTPWT, QQYVNYPFT, or QQYTSYPFT.

8. The antigen binding protein of any one of claims 1 to 7 wherein CDRH3 comprises the amino acid sequence, PFAY.

9. The antigen binding protein of anyone of claims 1 to 8 wherein said polypeptide is a Fab or F(ab)₂ fragment.

10. The antigen binding protein of claim 1, wherein the first immunoglobulin variable domain is a single chain variable domain.

11. The antigen binding protein of anyone of claims 1 to 10 wherein said antigen binding protein further comprises a second immunoglobulin variable domain, capable of binding to a second antigen.

12. The antigen binding protein of claim 11 wherein said second immunoglobulin variable domain binds to at least one an antigen selected from the group of: human serum albumin, ADAMTS4, NGF, OSM, TNF-α, IL-6, VIP, TRPV1, TRPV4, ADAMTS1, Aggrecan, Collagen II, RANKL, Syndecan 4, Hedgehog, and/or IL-1.

13. The antigen binding protein of any one of claims 1 to 12 characterized by a dissociation constant KD(off) equal or less than about 2.5×10⁻¹⁰ M for human ADAMTS5.

14. The antigen binding protein of anyone of claims 1 to 13 wherein said antigen binding protein blocks and/or reduces at least one activity of ADAMTS5.

15. The antigen binding protein of anyone of claims 1 to 14 wherein said antigen binding protein blocks and/or reduces the cleavage of aggrecan by ADAMTS5 at the Glu³⁷³-Ala³⁷⁴ cleavage site.

16. The antigen binding protein of anyone of claims 1 to 15 wherein said antigen binding protein is capable of penetrating cartilage when administered to an animal.

17. An isolated polynucleotide encoding an antigen binding protein of anyone of claims 1 to 16.

18. A host cell comprising the polynucleotide of claim 17.

19. A pharmaceutical composition comprising at least one of the antigen binding proteins of any one of claims 1 to 16.

20. A pharmaceutical composition comprising a first antigen binding protein of any one of claims 1 to 16 and a second monoclonal antibody.

21. The pharmaceutical composition of claim 20 wherein said second monoclonal antibody binds an antigen selected from the group of ADAMTS4, ADAMTS5, NGF, OSM, TNF-α, IL-6, VIP, TRPV1, TRPV4, ADAMTS1, Aggrecan, Collagen II, RANKL, and/or IL-1.

22. A method of treating a patient in need thereof comprising administering at least one dose of pharmaceutical composition of any one of claims 19 to 21 to said patient.

23. The method of claim 22 wherein the patient is suffering from a disease of the cartilage.

24. The method of claim 23 wherein the patient is suffering from at least one disease selected from the group of: cancer, pain, chronic pain, neuropathic pain, postoperative pain, osteoarthritis, sports injuries, erosive arthritis, rheumatoid arthritis, psoriatic arthritis, Lyme arthritis, juvenile arthritis, ankylosing spondylosis, neuralgia, neuropathies, algesia, nerve injury, ischaemia, neurodegeneration, inflammatory diseases, cartilage degeneration, diseases affecting the larynx, trachea, auditory canal, intervertebral discs, ligaments, tendons, joint capsules or bone development, invertebral disc degeneration, osteopenia, or periodontal diseases, acute joint injury, and/or a disease related to joint destruction.

25. The method of claim 24, wherein the patient is suffering from osteoarthritis.

26. The method of any one of claims 22 to 25 wherein administering at least one dose of said pharmaceutical composition reduces cartilage degradation in said patient.

27. The method of any one of claims 22 to 26, wherein administering at least one dose of said pharmaceutical composition inhibits and/or reduces aggrecan cleavage in said patient.

28. The method of any one of claims 22 to 27, wherein said pharmaceutical composition is administered intravenously, intramuscularly, intraarticularly, subcutaneously, orally, intranasally, and/or via respiratory inhaler.

29. An isolated monoclonal antibody comprising six CDRs wherein CDRH1 is DAWMD (SEQ ID NO:2), CDRH2 is EIRNKANNHARHYAESVKG (SEQ ID NO:13), and CDRH3 is TYYYGSSYGYCDV (SEQ ID NO:18) and CDRL1 is RTSENIYSYLA (SEQ ID NO:20), CDRL2 is NAKTLAE (SEQ ID NO:22) and CDRL3 is QHHYGTPWT (SEQ ID NO:27).

30. An isolated monoclonal antibody comprising six CDRs wherein CDRH1 is DAWMD (SEQ ID NO:2), CDRH2 is EIRHKANDHAIFYDESVKG (SEQ ID NO:15), and CDRH3 is PFAY (SEQ ID NO:5) and CDRL1 is KASQSVGTTIV (SEQ ID NO:19), CDRL2 is SASNRHT (SEQ ID NO:23) and CDRL3 is QQYTSYPFT (SEQ ID NO:29).

31. An isolated monoclonal antibody or antigen-binding fragment thereof, wherein said antibody or antigen binding fragment thereof competitively binds to human ADAMTS5 with an antibody of claim 29 or claim 30.

32. An isolated monoclonal antibody or antigen-binding fragment thereof, wherein said antibody or antigen binding fragment thereof binds to a neutralizing epitope of human ADAMTS5 with an affinity of at least about 5×10⁴ liter/mole as measured by an association constant (Ka).

33. The antibody or antigen-binding fragment thereof of any one of claims 29 to 31 wherein said antibody comprises a human constant region.

34. The antibody or antigen-binding fragment of claim 1, which is of immunoglobulin class IgG1, IgG2, IgG3, IgG4 or IgM.

35. The antigen-binding fragment of any one of claims 29 to 34, wherein said fragment is selected from the group consisting of Fab, Fab′, F(ab′)₂ and Fv.

36. An antigen binding protein or a fragment thereof comprising an antibody V_{H} domain comprising an amino acid sequence selected from the group consisting of SEQ ID NO: 76, 80, 116, 118, 120, 122, 124, 126, 128, 136, 138, 140, 142, and 144.

37. An antigen binding protein or a fragment thereof comprising an antibody an antibody V_{L} domain comprising an amino acid sequence selected from the group consisting of SEQ ID NO: 78, 82, 130, 132, 134, and 146.

38. An antigen binding protein or a fragment thereof comprising an antibody V_{H} domain of claim 36 and a V_{L} domain of claim 37.

39. The antigen binding protein or a fragment thereof of claim 38 comprising an antibody V_{H} domain comprising SEQ ID NO: 76 and a V_{L} domain comprising SEQ ID NO: 78.

40. The antigen binding protein or a fragment thereof of claim 38 comprising an antibody V_{H} domain comprising SEQ ID NO: 80 and a V_{L} domain comprising SEQ ID NO: 82.

41. The antigen binding protein or a fragment thereof of claim 38 comprising an antibody V_{H} domain comprising an amino acid sequence selected from the group consisting of SEQ ID NO: 116, 118, 120, 122, 124, 126, and 128 and a V_{L} domain comprising an amino acid sequence selected from the group consisting of SEQ ID NO: 130, 132, and 134.

42. The antigen binding protein or a fragment thereof of claim 38 comprising an antibody V_{H} domain comprising an amino acid sequence selected from the group consisting of SEQ ID NO: 136, 138, 140, 142, and 144 and a V_{L} domain comprising SEQ ID NO: 146.

43. An antigen binding protein or a fragment thereof comprising an antibody heavy chain comprising an amino acid sequence selected from the group consisting of SEQ ID NO: 68, 72, 84, 86, 88, 90, 92, 94, 96, 104, 106, 108, 110, 112, and 158.

44. An antigen binding protein or a fragment thereof comprising an antibody light chain comprising an amino acid sequence selected from the group consisting of SEQ ID NO: 70, 74, 98, 100, 102, and 114.

45. An antigen binding protein or a fragment thereof comprising an antibody heavy chain of claim 43 comprising an amino acid sequence selected from the group consisting of SEQ and an antibody light chain of claim 44.

46. The antigen binding protein or a fragment thereof of claim 45 comprising an antibody heavy chain comprising SEQ ID NO: 68 and an antibody light chain comprising SEQ ID NO: 70.

47. The antigen binding protein or a fragment thereof of claim 45 comprising an antibody heavy chain comprising SEQ ID NO: 72 and an antibody light chain comprising SEQ ID NO: 74.

48. The antigen binding protein or a fragment thereof of claim 45 comprising an antibody heavy chain comprising an amino acid sequence selected from the group consisting of SEQ ID NO: 84, 86, 88, 90, 92, 94, and 96 and an antibody light chain comprising an amino acid sequence selected from the group consisting of SEQ ID NO: 98, 100, and 102.

49. The antigen binding protein or a fragment thereof of claim 45 comprising an antibody heavy chain comprising an amino acid sequence selected from the group consisting of SEQ ID NO: 104, 106, 108, 110, 112, and 158 and an antibody light chain comprising SEQ ID NO: 114.

50. An isolated polynucleotide encoding an antibody V_{H} domain comprising an amino acid sequence selected from the group consisting of SEQ ID NO: 76, 80, 116, 118, 120, 122, 124, 126, 128, 136, 138, 140, 142, and 144.

51. The isolated polynucleotide of claim 50 wherein said polynucleotide is selected from the group consisting of SEQ ID NO: 75, 79, 115, 117, 119, 121, 123, 125, 127, 135, 137, 139, 141, 143, and 159.

52. An isolated polynucleotide encoding an antibody V_{L} domain comprising an amino acid sequence selected from the group consisting of SEQ ID NO: 78, 82, 130, 132, 134, and 146.

53. The isolated polynucleotide of claim 52 wherein said polynucleotide is selected from the group consisting of SEQ ID NO: 77, 81, 129, 131, 133, and 145.

54. An isolated polynucleotide encoding an antibody heavy chain comprising an amino acid sequence selected from the group consisting of SEQ ID NO: 68, 72, 84, 86, 88, 90, 92, 94, 96, 104, 106, 108, 110, 112, and 158.

55. The isolated polynucleotide of claim 54 wherein said polynucleotide is selected from the group consisting of SEQ ID NO: 67, 71, 83, 85, 87, 89, 91, 93, 95, 103, 105, 107, 109, 111, and 159.

56. An isolated polynucleotide encoding an antibody light chain comprising an amino acid sequence selected from the group consisting of SEQ ID NO: 70, 74, 98, 100, 102, and 114.

57. The isolated polynucleotide of claim 56 wherein said polynucleotide is selected from the group consisting of SEQ ID NO: 69, 73, 97, 99, 101, and 115.

58. A method of treating a patient in need thereof comprising administering at least one antigen binding protein of any one of claims 1 to 16 to said patient.

59. An isolated monoclonal antibody or antigen-binding fragment thereof, wherein said antibody or antigen binding fragment thereof binds to a neutralizing epitope of human ADAMTS5 with a dissociation constant (Kd) of less than about 5×10⁻⁴ liter/second.


SEQUENCE LISTING (159 sequences)

SEQ ID NO: 1 - protein, 7 aa, Artificial Sequence
  CDR
ARGSVIL

SEQ ID NO: 2 - protein, 5 aa, Artificial Sequence
  CDR
DAWMD

SEQ ID NO: 3 - protein, 19 aa, Artificial Sequence
  CDR
EIRHKANDHAIFYXESVKG

SEQ ID NO: 4 - protein, 13 aa, Artificial Sequence
  CDR
TYYYGSSYGYCDV

SEQ ID NO: 5 - protein, 4 aa, Artificial Sequence
  CDR
PFAY

SEQ ID NO: 6 - protein, 11 aa, Artificial Sequence
  CDR
KASQSVGTTIV

SEQ ID NO: 7 - protein, 11 aa, Artificial Sequence
  CDR
RTSENIYSYLA

SEQ ID NO: 8 - protein, 7 aa, Artificial Sequence
  CDR
NAKTLAE

SEQ ID NO: 9 - protein, 7 aa, Artificial Sequence
  CDR
SASNRXT

SEQ ID NO: 10 - protein, 9 aa, Artificial Sequence
  CDR
QQYSSYPFT

SEQ ID NO: 11 - protein, 9 aa, Artificial Sequence
  CDR
QHHYGTPWT

SEQ ID NO: 12 - protein, 19 aa, Artificial Sequence
  CDR
EIRHKANDHAIFYAESVKG

SEQ ID NO: 13 - protein, 19 aa, Artificial Sequence
  CDR
EIRNKANNHARHYAESVKG

SEQ ID NO: 14 - protein, 19 aa, Artificial Sequence
  CDR
EIRHKANDYAIFYDESVKG

SEQ ID NO: 15 - protein, 19 aa, Artificial Sequence
  CDR
EIRHKANDHAIFYDESVKG

SEQ ID NO: 16 - protein, 19 aa, Artificial Sequence
  CDR
DIRNTANNHATFYAESVKG

SEQ ID NO: 17 - protein, 19 aa, Artificial Sequence
  CDR
EIRHKANDHAIFYDESVKG

SEQ ID NO: 18 - protein, 13 aa, Artificial Sequence
  CDR
TYYYGSSYGYCDV

SEQ ID NO: 19 - protein, 11 aa, Artificial Sequence
  CDR
KASQSVGTTIV

SEQ ID NO: 20 - protein, 11 aa, Artificial Sequence
  CDR
RTSENIYSYLA

SEQ ID NO: 21 - protein, 11 aa, Artificial Sequence
  CDR
KASQNVGTAVV

SEQ ID NO: 22 - protein, 7 aa, Artificial Sequence
  CDR
NAKTLAE

SEQ ID NO: 23 - protein, 7 aa, Artificial Sequence
  CDR
SASNRHT

SEQ ID NO: 24 - protein, 7 aa, Artificial Sequence
  CDR
SASTRYT

SEQ ID NO: 25 - protein, 7 aa, Artificial Sequence
  CDR
SASNRYT

SEQ ID NO: 26 - protein, 9 aa, Artificial Sequence
  CDR
QQYSSYPFT

SEQ ID NO: 27 - protein, 9 aa, Artificial Sequence
  CDR
QHHYGTPWT

SEQ ID NO: 28 - protein, 9 aa, Artificial Sequence
  CDR
QQYVNYPFT

SEQ ID NO: 29 - protein, 9 aa, Artificial Sequence
  CDR
QQYTSYPFT

SEQ ID NO: 30 - protein, 115 aa, Artificial Sequence
  Heavy chain
EVKLEESGGGLVQPGGSMKLSCAASGFTFSDAWMDWVRQSPEKGLEWVAEIRHKANDHAIFYXESVKGRFTISRDDSKSIVYLQMNSLRPEDTGIYYCTSPFAYWGQGTLVTVSA

SEQ ID NO: 31 - protein, 109 aa, Artificial Sequence
  Light chain
DIVMTQSQKFMSVTVGDRVSITCKASQSVGTTIVWYQQKPGQSPKLLIYSASNRXTGVPDRFTGSGSGTDFILTINNVQSEDLADYFCQQYSSYPFTFGSGTKLEIKRA

SEQ ID NO: 32 - protein, 115 aa, Artificial Sequence
  Heavy chain
EVKLEESGGGLVQPGGSMKLSCTASGFTFSDAWMDWVRQSPEKGLEWVAEIRHKANDHAIFYDESVKGRFTISRDDSKNIVYLQMNSLRPEDTGIYYCTSPFAYWGQGTLVTVSA

SEQ ID NO: 33 - protein, 115 aa, Artificial Sequence
  Heavy chain
EVKLEESGGGLGQPGGSMKLSCAASGFNFSDAWMDWVRQSPEKGLEWVAEIRHKANDHAIFYAESVKGRFTISRDDSKSSVYLQMNSLRAEDTGIYYCTSPFAYWGQGTLVTVSA

SEQ ID NO: 34 - protein, 115 aa, Artificial Sequence
  Heavy chain
EVKLEESGGGLVQPGGSMKLSCAASGITFSDAWMDWVRQSPEKGLEWVADIRNTANNHATFYAESVKGRFTISRDDSKSTVYLQMNTLRPEDTGIYYCTSPFAYWGQGTLVTVSA

SEQ ID NO: 35 - protein, 115 aa, Artificial Sequence
  Heavy chain
EVKFEESGGGLVQPGGSMKLSCAASGFIFSDAWMDWVRQSPEKGLEWVAEIRHKANDYAIFYDESVKGRFTISRDDSKSIVYLQMNNLRPEDTGIYYCTSPFAYWGQGTLVTVSA

SEQ ID NO: 36 - protein, 115 aa, Artificial Sequence
  Heavy chain
EVKLEESGGGLVQPGGSMKLSCAASGFTFSDAWMDWVRQSPEKGLEWVAEIRHKANDHAIFYDESVKGRFTISRDDSKSIVYLQMNSLRPEDTGIYYCTSPFAYWGQGTLVTVSA

SEQ ID NO: 37 - protein, 124 aa, Artificial Sequence
  Heavy chain
EVKLEESGGGLVQPGGSMKLSCAASGFTSSDAWMDWVRQSPEKGLEWVAEIRNKANNHARHYAESVKGRFTISRDDSKSSVYLQMNSLRAEDSGIYYCTRTYYYGSSYGYCDVWGTGTTVTVSS

SEQ ID NO: 38 - protein, 109 aa, Artificial Sequence
  Light chain
DIVMTQSQKFMSTTVGDRVSITCKASQNVGTAVVWFQQKPGQSPKLLIYSASNRYTRVPDRFTGSGSGTDFTLTISNVQSEDLADYFCQQYVNYPFTFGSGTKLEIKRA

SEQ ID NO: 39 - protein, 109 aa, Artificial Sequence
  Light chain
DIVMTQSQKFMSVTVGDRVSITCKASQSVGTTIVWYQQKPGQSPKLLIYSASNRHTGVPDRFTGSGSGTDFILTISNVQSEDLADYFCQQYSSYPFTFGSGTKLEIKRA

SEQ ID NO: 40 - protein, 109 aa, Artificial Sequence
  Light chain
DIVMTQSQRFMSVTVGDRVSITCKASQSVGTTIVWYQQKPGQSPKLLIYSASTRYTGVPDRFTGGGSGTDFILTINNVQSEDLADYFCQQYSSYPFTFGSGTKLEIKRA

SEQ ID NO: 41 - protein, 109 aa, Artificial Sequence
  Light chain
DIQMTQSPASLSASVGETVTITCRTSENIYSYLAWYQQKQGKSPQLLVYNAKTLAEGVPSRFSGSGSGTQFSLKINSLQPEDFGSYSCQHHYGTPWTFGGGTKLEIKRA

SEQ ID NO: 42 - protein, 109 aa, Artificial Sequence
  Light chain
DIVMTQSQKFMSVTVGDRVSITCKASQSVGTTIVWYQQKPGQSPKLLIYSASNRHTGVPDRFTGSGSGTDFILTINNVQSEDLADYFCQQYTSYPFTFGSGTKLEIKRA

SEQ ID NO: 43 - protein, 930 aa, Homo sapien
MLLGWASLLLCAFRLPLAAVGPAATPAQDKAGQPPTAAAAAQPRRRQGEEVQERAEPPGHPHPLAQRRRSKGLVQNIDQLYSGGGKVGYLVYAGGRRFLLDLERDGSVGIAGFVPAGGGTSAPWRHRSHCFYRGTVDGSPRSLAVFDLCGGLDGFFAVKHARYTLKPLLRGPWAEEEKGRVYGDGSARILHVYTREGFSFEALPPRASCETPASTPEAHEHAPAHSNPSGRAALASQLLDQSALSPAGGSGPQTWWRRRRRSISRARQVELLLVADASMARLYGRGLQHYLLTLASIANRLYSHASIENHIRLAVVKVVVLGDKDKSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEETFGSTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRICLQGKCVDKTKKKYYSTSSHGNWGSWGSWGQCSRSCGGGVQFAYRHCNNPAPRNNGRYCTGKRAIYRSCSLMPCPPNGKSFRHEQCEAKNGYQSDAKGVKTFVEWVPKYAGVLPADVCKLTCRAKGTGYYVVFSPKVTDGTECRLYSNSVCVRGKCVRTGCDGIIGSKLQYDKCGVCGGDNSSCTKIVGTFNKKSKGYTDVVRIPEGATHIKVRQFKAKDQTRFTAYLALKKKNGEYLINGKYMISTSETIIDINGTVMNYSGWSHRDDFLHGMGYSATKEILIVQILATDPTKPLDVRYSFFVPKKSTPKVNSVTSHGSNKVGSHTSQPQWVTGPWLACSRTCDTGWHTRTVQCQDGNRKLAKGCPLSQRPSAFKQCLLKKC

SEQ ID NO: 44 - protein, 837 aa, Homo sapien [sequence omitted]

SEQ ID NO: 45 - DNA, 57 bp, Artificial Sequence
  Signal sequence
atgggctggtcctgcatcatcctgtttctggtggccaccgccaccggcgtgcacagc

SEQ ID NO: 46 - protein, 19 aa, Artificial Sequence
  Signal sequence
MGWSCIILFLVATATGVHS

SEQ ID NO: 47 - DNA, 372 bp, Murine
gaagtgaagcttgaggagtctggaggaggcttggtgcaacctggaggatccatgaaactctcttgtgctgcctctggattcacttctagtgacgcctggatggactgggtccgccagtctccagaaaaggggcttgagtgggttgctgaaattagaaacaaagctaataatcatgcaagacactatgctgagtctgtgaaagggaggttcaccatctcaagagatgattccaaaagtagtgtctacctgcaaatgaacagcttaagagctgaagactctggcatttattactgtaccaggacgtattattacggtagcagctacggatactgcgatgtctggggcacagggaccacggtcaccgtctcctca

SEQ ID NO: 48 - protein, 124 aa, Murine; identical to SEQ ID NO: 37

SEQ ID NO: 49 - DNA, 327 bp, Murine
gacatccagatgactcagtctccagcctccctatctgcatctgtgggagaaactgtcaccatcacatgtcgaacaagtgagaatatttacagttatttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattcctgtcaacatcattatggtactccgtggacgttcggtggaggcaccaagctggaaatcaaacgggct

SEQ ID NO: 50 - protein, 109 aa, Murine; identical to SEQ ID NO: 41

SEQ ID NO: 51 - DNA, 345 bp, Murine
gaagtgaagcttgaggagtctggaggaggcttggtgcaacctggaggatccatgaaactctcctgtactgcctccggattcacttttagtgacgcctggatggactgggtccgccagtctccagagaagggacttgagtgggttgctgaaattagacacaaagctaatgatcatgcaatattttatgatgagtctgtgaaagggaggttcaccatctcaagagatgattccaaaaatattgtctatctgcaaatgaacagtttaagacctgaggacaccggcatttattattgtaccagtccttttgcttattggggccaagggactctggtcactgtctctgca

SEQ ID NO: 52 - protein, 115 aa, Murine; identical to SEQ ID NO: 32

SEQ ID NO: 53 - DNA, 327 bp, Murine
gacattgtgatgacccagtctcaaaaattcatgtccgtaacagtgggagacagggtcagcatcacctgcaaggccagtcagagtgtgggaactactatagtctggtatcaacagaaaccaggacaatctcctaaattactgatttactctgcatccaatcggcacactggggtccctgatcgcttcacaggcagtggatctgggacagatttcattctcaccattaacaatgtgcagtctgaggacctggcagattatttctgtcagcaatatacctcctatccattcacgttcggctcggggacaaagttggagataaaacgggct

SEQ ID NO: 54 - protein, 109 aa, Murine; identical to SEQ ID NO: 42

SEQ ID NO: 55 - protein, 5 aa, Artificial Sequence
  CDR
DAWMD

SEQ ID NO: 56 - protein, 19 aa, Artificial Sequence
  CDR
EIRNKANNHARHYAESVKG

SEQ ID NO: 57 - protein, 13 aa, Artificial Sequence
  CDR
TYYYGSSYGYCDV

SEQ ID NO: 58 - protein, 11 aa, Artificial Sequence
  CDR
RTSENIYSYLA

SEQ ID NO: 59 - protein, 7 aa, Artificial Sequence
  CDR
NAKTLAE

SEQ ID NO: 60 - protein, 9 aa, Artificial Sequence
  CDR
QHHYGTPWT

SEQ ID NO: 61 - protein, 5 aa, Artificial Sequence
  CDR
DAWMD

SEQ ID NO: 62 - protein, 19 aa, Artificial Sequence
  CDR
EIRHKANDHAIFYDESVKG

SEQ ID NO: 63 - protein, 4 aa, Artificial Sequence
  CDR
PFAY

SEQ ID NO: 64 - protein, 11 aa, Artificial Sequence
  CDR
KASQSVGTTIV

SEQ ID NO: 65 - protein, 7 aa, Artificial Sequence
  CDR
SASNRHT

SEQ ID NO: 66 - protein, 9 aa, Artificial Sequence
  CDR
QQYTSYPFT

SEQ ID NO: 67 - DNA, 1362 bp, Artificial Sequence
  Heavy chain
gaggtgaagctggaggaatcaggaggcggactggtgcagcccggcggcagcatgaaactgagctgcgctgcctctggcttcaccagctctgacgcctggatggattgggtgaggcagagccccgagaagggcctggagtgggtggccgagatcaggaacaaggccaacaaccacgccaggcactacgccgagagcgtgaagggcaggttcaccatcagcagggacgacagcaagagctccgtgtacctgcagatgaacagcctccgggccgaggacagcggcatctactactgcaccaggacctactactacgggagcagctacggctattgcgacgtgtggggaaccggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 68 - protein, 454 aa, Artificial Sequence
  Heavy chain
EVKLEESGGGLVQPGGSMKLSCAASGFTSSDAWMDWVRQSPEKGLEWVAEIRNKANNHARHYAESVKGRFTISRDDSKSSVYLQMNSLRAEDSGIYYCTRTYYYGSSYGYCDVWGTGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - DNA, 642 bp, Artificial Sequence
  Light chain
gacatccagatgacccagagccccgccagcctgagcgcaagcgtgggcgagaccgtcaccatcacctgcaggaccagcgagaacatctacagctacctggcctggtatcagcagaagcagggcaagagcccccagctgctggtgtacaacgccaaaaccctggccgaaggcgtgccctctaggttcagcggaagcggaagcggcacccagttcagcctcaagatcaactccctgcagcccgaggacttcggcagctacagctgccagcaccactacggcactccctggacctttggcggcggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 70 - protein, 214 aa, Artificial Sequence
  Light chain
DIQMTQSPASLSASVGETVTITCRTSENIYSYLAWYQQKQGKSPQLLVYNAKTLAEGVPSRFSGSGSGTQFSLKINSLQPEDFGSYSCQHHYGTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - DNA, 1335 bp, Artificial Sequence
  Heavy chain
gaggtgaaactggaggaaagcggcggcggactggtgcagcccggcggctcaatgaagctgagctgcaccgccagcggcttcaccttctctgacgcctggatggactgggtgaggcagagccccgagaagggcctggagtgggtggctgagatcaggcacaaggccaacgaccacgccatcttctacgacgagagcgtgaagggcaggttcaccatcagcagggacgacagcaagaacatcgtgtacctccagatgaacagcctgaggcccgaggataccggcatctactactgcaccagcccctttgcctattggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 72 - protein, 445 aa, Artificial Sequence
  Heavy chain
EVKLEESGGGLVQPGGSMKLSCTASGFTFSDAWMDWVRQSPEKGLEWVAEIRHKANDHAIFYDESVKGRFTISRDDSKNIVYLQMNSLRPEDTGIYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - DNA, 642 bp, Artificial Sequence
  Light chain
gacatcgtgatgacccagagccagaaattcatgagcgtgaccgtgggcgacagggtgtccatcacttgcaaggccagccagagcgtgggcaccactatcgtgtggtaccagcagaagcccggccagagccccaagctcctgatctacagcgccagcaacaggcacaccggagtgcccgacaggtttactggcagcggctcaggcaccgacttcatcctgaccatcaacaacgtgcagagcgaggacctggccgattacttctgccagcagtacaccagctaccccttcaccttcggctcagggaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 74 - protein, 214 aa, Artificial Sequence
  Light chain
DIVMTQSQKFMSVTVGDRVSITCKASQSVGTTIVWYQQKPGQSPKLLIYSASNRHTGVPDRFTGSGSGTDFILTINNVQSEDLADYFCQQYTSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 75 - DNA, 372 bp, Artificial Sequence
  Heavy chain
gaggtgaagctggaggaatcaggaggcggactggtgcagcccggcggcagcatgaaactgagctgcgctgcctctggcttcaccagctctgacgcctggatggattgggtgaggcagagccccgagaagggcctggagtgggtggccgagatcaggaacaaggccaacaaccacgccaggcactacgccgagagcgtgaagggcaggttcaccatcagcagggacgacagcaagagctccgtgtacctgcagatgaacagcctccgggccgaggacagcggcatctactactgcaccaggacctactactacgggagcagctacggctattgcgacgtgtggggaaccggcacactagtcaccgtgagcagc

SEQ ID NO: 76 - protein, 124 aa, Artificial Sequence
  Heavy chain
EVKLEESGGGLVQPGGSMKLSCAASGFTSSDAWMDWVRQSPEKGLEWVAEIRNKANNHARHYAESVKGRFTISRDDSKSSVYLQMNSLRAEDSGIYYCTRTYYYGSSYGYCDVWGTGTLVTVSS

SEQ ID NO: 77 - DNA, 327 bp, Artificial Sequence
  Light chain
gacatccagatgacccagagccccgccagcctgagcgcaagcgtgggcgagaccgtcaccatcacctgcaggaccagcgagaacatctacagctacctggcctggtatcagcagaagcagggcaagagcccccagctgctggtgtacaacgccaaaaccctggccgaaggcgtgccctctaggttcagcggaagcggaagcggcacccagttcagcctcaagatcaactccctgcagcccgaggacttcggcagctacagctgccagcaccactacggcactccctggacctttggcggcggcaccaagctggagattaagcgtacg

SEQ ID NO: 78 - protein, 109 aa, Artificial Sequence
  Light chain
DIQMTQSPASLSASVGETVTITCRTSENIYSYLAWYQQKQGKSPQLLVYNAKTLAEGVPSRFSGSGSGTQFSLKINSLQPEDFGSYSCQHHYGTPWTFGGGTKLEIKRT

SEQ ID NO: 79 - DNA, 345 bp, Artificial Sequence
  Heavy chain
gaggtgaaactggaggaaagcggcggcggactggtgcagcccggcggctcaatgaagctgagctgcaccgccagcggcttcaccttctctgacgcctggatggactgggtgaggcagagccccgagaagggcctggagtgggtggctgagatcaggcacaaggccaacgaccacgccatcttctacgacgagagcgtgaagggcaggttcaccatcagcagggacgacagcaagaacatcgtgtacctccagatgaacagcctgaggcccgaggataccggcatctactactgcaccagcccctttgcctattggggccagggcacactagtcaccgtgtccagc

SEQ ID NO: 80 - protein, 115 aa, Artificial Sequence
  Heavy chain
EVKLEESGGGLVQPGGSMKLSCTASGFTFSDAWMDWVRQSPEKGLEWVAEIRHKANDHAIFYDESVKGRFTISRDDSKNIVYLQMNSLRPEDTGIYYCTSPFAYWGQGTLVTVSS

SEQ ID NO: 81 - DNA, 327 bp, Artificial Sequence
  Light chain
gacatcgtgatgacccagagccagaaattcatgagcgtgaccgtgggcgacagggtgtccatcacttgcaaggccagccagagcgtgggcaccactatcgtgtggtaccagcagaagcccggccagagccccaagctcctgatctacagcgccagcaacaggcacaccggagtgcccgacaggtttactggcagcggctcaggcaccgacttcatcctgaccatcaacaacgtgcagagcgaggacctggccgattacttctgccagcagtacaccagctaccccttcaccttcggctcagggaccaagctggagattaagcgtacg

SEQ ID NO: 82 - protein, 109 aa, Artificial Sequence
  Light chain
DIVMTQSQKFMSVTVGDRVSITCKASQSVGTTIVWYQQKPGQSPKLLIYSASNRHTGVPDRFTGSGSGTDFILTINNVQSEDLADYFCQQYTSYPFTFGSGTKLEIKRT

SEQ ID NO: 83 - DNA, 1362 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 84 - protein, 454 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - DNA, 1362 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 86 - protein, 454 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 88 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCARTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 90 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcctgtacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 92 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSLYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcgtctacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 94 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSVYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - DNA, 1362 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcgtctacctgcagatgaacagcctgagggccgaggacaccgccgtgtactactgcacccggacctactactacgggagcagctacggctattgcgacgtgtggggccagggcacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 96 - protein, 454 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSVYLQMNSLRAEDTAVYYCTRTYYYGSSYGYCDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - DNA, 642 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctgatctacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 98 - protein, 214 aa, Artificial Sequence
  Light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - DNA, 642 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 100 - protein, 214 aa, Artificial Sequence
  Light chain
DIQMTQSPSSLSASVGDRVTITCRTSENIYSYLAWYQQKPGKAPKLLVYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - DNA, 642 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggccccccagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 102 - protein, 214 aa, Artificial Sequence
  Light chain
DIQMTQSPSSLSASVGDRVTITCRTSENIYSYLAWYQQKPGKAPQLLVYNAKTLAEGVPSRFSGSGSGTDFTLTISSLQPEDFATYYCQHHYGTPWTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcgccaagccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 104 - protein, 445 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCAKPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 106 - protein, 445 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacgacagcaagaacaccctgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 108 - protein, 445 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDDSKNTLYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacaacagcaagaacaccgtgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 110 - protein, 445 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDNSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacgacagcaagaacaccgtgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctgctgggaggccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag

SEQ ID NO: 112 - protein, 445 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 642 bp, Artificial Sequence
  Light chain
gacatccagatgacccagagcccctcaagcctgtctgccagcgtgggcgacagggtgaccattacctgcaaggccagccagagcgtcggcaccaccatcgtgtggtaccagcagaaacccggcaaggcccccaagctcctgatctacagcgccagcaacaggcacaccggcgtgcctagcaggtttagcggaagcggcagcggcaccgacttcaccctgacaatcagcagcctgcagcccgaggacttcgccacctactactgccagcagtacaccagctatcccttcaccttcggccagggcaccaagctggagatcaagcgtacggtggccgcccccagcgtgttcatcttcccccccagcgatgagcagctgaagagcggcaccgccagcgtggtgtgtctgctgaacaacttctacccccgggaggccaaggtgcagtggaaggtggacaatgccctgcagagcggcaacagccaggagagcgtgaccgagcaggacagcaaggactccacctacagcctgagcagcaccctgaccctgagcaaggccgactacgagaagcacaaggtgtacgcctgtgaggtgacccaccagggcctgtccagccccgtgaccaagagcttcaaccggggcgagtgc

SEQ ID NO: 114 - protein, 214 aa, Artificial Sequence
  Light chain
DIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - DNA, 372 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagc

SEQ ID NO: 116 - protein, 124 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCARTYYYGSSYGYCDVWGQGTLVTVSS

SEQ ID NO: 117 - DNA, 372 bp, Artificial Sequence
  Heavy chain
gaggtgcagctggtcgaaagcggcggcggcctggtgcagcccggaggcagcctcaggctgtcttgcgctgcctccggcttcaccttcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagc

SEQ ID NO: 118 - protein, 124 aa, Artificial Sequence
  Heavy chain
EVQLVESGGGLVQPGGSLRLSCAASGFTFSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSS

SEQ ID NO: 119 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcgcccggacctactactacgggagcagctacggctattgcgacgtgtggggccagggcacactagtgaccgtgtccagc

SEQ ID NO: 120 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCARTYYYGSSYGYCDVWGQGTLVTVSS

SEQ ID NO: 121 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcctgtacctgcagatgaacagcctgagggccgaggacaccgccgtgtactactgcacccggacctactactacgggagcagctacggctattgcgacgtgtggggccagggcacactagtgaccgtgtccagc

SEQ ID NO: 122 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSLYLQMNSLRAEDTAVYYCTRTYYYGSSYGYCDVWGQGTLVTVSS

SEQ ID NO: 123 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcctgtacctgcagatgaacagcctgagggccgaggacaccgccgtgtactactgcacccggacctactactacgggagcagctacggctattgcgacgtgtggggccagggcacactagtgaccgtgtccagc

SEQ ID NO: 124 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSLYLQMNSLRAEDTAVYYCTRTYYYGSSYGYCDVWGQGTLVTVSS

SEQ ID NO: 125 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacaacgccaagaacagcgtctacctgcagatgaacagcctgagggccgaggacaccgccgtgtactactgcacccggacctactactacgggagcagctacggctattgcgacgtgtggggccagggcacactagtgaccgtgtccagc

SEQ ID NO: 126 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDNAKNSVYLQMNSLRAEDTAVYYCTRTYYYGSSYGYCDVWGQGTLVTVSS

SEQ ID NO: 127 - DNA, 372 bp, Artificial Sequence
  Heavy chain
gaggtgcagctggtcgaaagcggcggcggcctggtgcagcccggaggcagcctcaggctgtcttgcgctgcctccggcttcaccagcagcgacgcctggatggattgggtgaggcaggcccccggcaaaggcctggagtgggtggccgagatcaggaacaaggccaacaaccacgccaggcactacgccgagagcgtgaaggggaggttcaccatcagcagggacgacgccaagaacagcgtctacctgcagatgaacagcctgagggccgaggacaccgccgtgtactactgcacccggacctactactacgggagcagctacggctattgcgacgtgtggggccagggcacactagtgaccgtgtccagc

SEQ ID NO: 128 - protein, 124 aa, Artificial Sequence
  Heavy chain
EVQLVESGGGLVQPGGSLRLSCAASGFTSSDAWMDWVRQAPGKGLEWVAEIRNKANNHARHYAESVKGRFTISRDDAKNSVYLQMNSLRAEDTAVYYCTRTYYYGSSYGYCDVWGQGTLVTVSS

SEQ ID NO: 129 - DNA, 327 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctgatctacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacg

SEQ ID NO: 130 - protein, 109 aa, Artificial Sequence
  Light chain
DIQMTQSPSSLSASVGDRVTITCRTSENIYSYLAWYQQKPGKAPKLLIYNAKTLAEGVPSRFSGSGSGTDFTLTISSLQPEDFATYYCQHHYGTPWTFGQGTKLEIKRT

SEQ ID NO: 131 - DNA, 327 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggcccccaagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacg

SEQ ID NO: 132 - protein, 109 aa, Artificial Sequence
  Light chain
DIQMTQSPSSLSASVGDRVTITCRTSENIYSYLAWYQQKPGKAPKLLVYNAKTLAEGVPSRFSGSGSGTDFTLTISSLQPEDFATYYCQHHYGTPWTFGQGTKLEIKRT

SEQ ID NO: 133 - DNA, 327 bp, Artificial Sequence
  Light chain
gacatccagatgacccagagccccagcagcctgagcgctagcgtgggcgacagggtgaccatcacctgcaggacctccgagaacatctacagctacctggcctggtatcagcagaaacccggcaaggccccccagctcctggtgtacaacgccaagacactggccgagggcgtgcccagcaggttctcaggcagcggctctgggaccgacttcaccctgaccatcagcagcctgcagcccgaggacttcgccacctactactgccagcaccactacggaaccccctggaccttcggccagggcaccaagctggagattaagcgtacg

SEQ ID NO: 134 - protein, 109 aa, Artificial Sequence
  Light chain
DIQMTQSPSSLSASVGDRVTITCRTSENIYSYLAWYQQKPGKAPQLLVYNAKTLAEGVPSRFSGSGSGTDFTLTISSLQPEDFATYYCQHHYGTPWTFGQGTKLEIKRT

SEQ ID NO: 135 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcgccaagccctttgcctactggggccagggaacactagtgaccgtgtccagc

SEQ ID NO: 136 - protein, 115 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCAKPFAYWGQGTLVTVSS

SEQ ID NO: 137 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacaacagcaagaacaccctgtacctgcagatgaacagcctgagggccgaagacaccgctgtgtactactgcaccagcccctttgcctactggggccagggaacactagtgaccgtgtccagc

SEQ ID NO: 138 - protein, 115 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDNSKNTLYLQMNSLRAEDTAVYYCTSPFAYWGQGTLVTVSS

SEQ ID NO: 139 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcagcgagatcaggcacaaggccaacgaccacgccatcttctacgacgagagcgtgaagggcaggttcaccatctccagggacgacagcaagaacaccctgtacctgcagatgaacagcctgagggccgaagacaccgctgtgtactactgcaccagcccctttgcctactggggccagggaacactagtgaccgtgtccagc

SEQ ID NO: 140 - protein, 115 aa, Artificial Sequence
  Heavy chain
EVQLLESGGGLVQPGGSLRLSCAASGFTFSDAWMDWVRQAPGKGLEWVSEIRHKANDHAIFYDESVKGRFTISRDDSKNTLYLQMNSLRAEDTAVYYCTSPFAYWGQGTLVTVSS

SEQ ID NO: 141 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacaacagcaagaacaccgtgtacctgcagatgaacagcctgagggccgaagacaccgctgtgtactactgcaccagcccctttgcctactggggccagggaacactagtgaccgtgtccagc

SEQ ID NO: 142 - protein, 115 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDNSKNTVYLQMNSLRAEDTAVYYCTSPFAYWGQGTLVTVSS

SEQ ID NO: 143 - DNA, 345 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacgacagcaagaacaccgtgtacctgcagatgaacagcctgagggccgaagacaccgctgtgtactactgcaccagcccctttgcctactggggccagggaacactagtgaccgtgtccagc

SEQ ID NO: 144 - protein, 115 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSS

SEQ ID NO: 145 - DNA, 327 bp, Artificial Sequence
  Light chain
gacatccagatgacccagagcccctcaagcctgtctgccagcgtgggcgacagggtgaccattacctgcaaggccagccagagcgtcggcaccaccatcgtgtggtaccagcagaaacccggcaaggcccccaagctcctgatctacagcgccagcaacaggcacaccggcgtgcctagcaggtttagcggaagcggcagcggcaccgacttcaccctgacaatcagcagcctgcagcccgaggacttcgccacctactactgccagcagtacaccagctatcccttcaccttcggccagggcaccaagctggagatcaagcgtacg

SEQ ID NO: 146 - protein, 109 aa, Artificial Sequence
  Light chain
DIQMTQSPSSLSASVGDRVTITCKASQSVGTTIVWYQQKPGKAPKLLIYSASNRHTGVPSRFSGSGSGTDFTLTISSLQPEDFATYYCQQYTSYPFTFGQGTKLEIKRT

SEQ ID NO: 147 - DNA, 345 bp, Artificial Sequence; identical to SEQ ID NO: 51
  Heavy chain

SEQ ID NO: 148 - DNA, 327 bp, Artificial Sequence; identical to SEQ ID NO: 53
  Light chain

SEQ ID NO: 149 - DNA, 372 bp, Artificial Sequence; identical to SEQ ID NO: 47
  Heavy chain

SEQ ID NO: 150 - DNA, 327 bp, Artificial Sequence; identical to SEQ ID NO: 49
  Light chain

SEQ ID NO: 151 - DNA, 345 bp, Artificial Sequence
  Heavy chain
gaagtgaagcttgaggagtctggaggaggcttggtgcaacctggaggatccatgaaactctcttgtgctgcctctggaatcacttttagtgatgcctggatggactgggtccgccagtctccagagaaggggcttgagtgggttgctgacattagaaacacagctaataatcatgcaacattctatgctgagtctgtgaaagggaggttcaccatctcaagagatgattccaaaagtactgtctacctgcaaatgaacaccttaagacctgaagacactggcatttattactgtaccagcccttttgcttactggggccaagggactctggtcactgtctctgca

SEQ ID NO: 152 - DNA, 327 bp, Artificial Sequence
  Light chain
gacattgtgatgacccagtctcaaaaattcatgtccacaacagtaggagacagggtcagcatcacctgcaaggccagtcaaaatgtgggtactgctgtagtctggtttcaacagaaaccaggacaatctcctaaactactgatttactcagcatccaatcggtacaccagagtccctgatcgcttcacaggcagtggatctgggacagatttcactctcaccattagcaatgtgcagtctgaagacctggcagattatttctgtcagcaatatgtcaactatccattcacgttcggctcggggacaaaattggaaataaaacgggct

SEQ ID NO: 153 - DNA, 345 bp, Artificial Sequence
  Heavy chain
gaagtgaagtttgaggagtctggaggaggcttggtgcaacctggaggatccatgaaactctcctgtgctgcctccggatttatttttagtgacgcctggatggactgggtccgccagtctccagagaagggacttgagtgggttgctgaaattagacacaaagctaatgattatgcaatattttatgatgagtctgtgaaagggaggttcaccatctcaagagatgattccaaaagtattgtctatctgcaaatgaacaacttaagacctgaagacaccggcatttattattgtaccagtccttttgcttactggggccaagggactctggtcactgtctctgca

SEQ ID NO: 154 - DNA, 327 bp, Artificial Sequence
  Light chain
gacattgtgatgacccagtctcaaaaattcatgtccgtaacagtgggagacagggtcagcatcacctgcaaggccagtcagagtgtgggaactactatagtctggtatcaacagaaaccaggacaatctcctaaattactgatttactcagcatccaatcgacacactggagtccctgatcgcttcacaggcagtggatctgggacagatttcattctcaccattagcaatgtgcagtctgaagacctggcagattatttctgtcagcaatatagcagctatccattcacgttcggctcggggacaaagttggaaataaaacgggct

SEQ ID NO: 155 - DNA, 345 bp, Artificial Sequence
  Heavy chain
gaggtgaagcttgaggagtctggaggaggcttggtgcaacctggaggatccatgaaactctcctgtgctgcctctggattcacttttagtgacgcctggatggactgggtccgccagtctccagagaagggacttgagtgggttgctgaaattagacacaaagctaatgatcatgcaatattttatgatgagtctgtgaaagggaggttcaccatctcaagagatgattccaaaagtattgtctatctgcaaatgaacagcttaagacctgaagacaccggcatttattattgtaccagtccttttgcttactggggccaagggactctggtcactgtctctgca

SEQ ID NO: 156 - DNA, 327 bp, Artificial Sequence
  Light chain
gacattgtgatgacccagtctcaaagattcatgtccgtaacagtgggagacagggtcagcatcacctgcaaggccagtcagagtgtgggtactactatagtctggtatcaacagaaaccaggacaatctcctaaactactgatttactcagcatccactcggtatactggggtccctgatcgcttcacaggcggtggatctgggacagatttcattctcaccattaacaatgtgcagtctgaagacctggcagattatttctgtcagcagtatagcagctatccattcacgttcggctcggggacaaagttggaaataaaacgggct

SEQ ID NO: 157 - DNA, 345 bp, Artificial Sequence
  Heavy chain
gaagtgaagcttgaggagtctggaggaggcttggggcaacctggaggatccatgaaactctcttgtgctgcctctggattcaattttagtgacgcctggatggactgggtccgccagtctccagaaaaggggcttgagtgggttgctgaaattagacacaaagctaatgatcatgcaatattctatgctgagtctgtgaaagggaggttcaccatctcaagagatgattccaaaagtagtgtctacctccaaatgaacagcttaagagctgaagacactggcatttattactgtaccagcccttttgcttactggggccaagggactctggtcactgtctctgca

SEQ ID NO: 158 - protein, 445 aa, Artificial Sequence
  Heavy chain
EVQLLESGGGLVQPGGSLRLSCAASGFTFSDAWMDWVRQAPGKGLEWVAEIRHKANDHAIFYDESVKGRFTISRDDSKNTVYLQMNSLRAEDTAVYYCTSPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - DNA, 1335 bp, Artificial Sequence
  Heavy chain
gaggtgcagctcctggagagcggcgggggcctggtgcagcccggcggcagcctgagactgagctgcgccgcaagcggcttcaccttcagcgacgcctggatggattgggtgaggcaggcccccggcaaaggactggagtgggtcgccgagatcaggcacaaggccaacgaccacgccatcttctacgacgagagcgtgaagggcaggttcaccatctccagggacgacagcaagaacaccgtgtacctgcagatgaacagcctgagggccgaagacaccgctgtgtactactgcaccagcccctttgcctactggggccagggaacactagtgaccgtgtccagcgccagcaccaagggccccagcgtgttccccctggcccccagcagcaagagcaccagcggcggcacagccgccctgggctgcctggtgaaggactacttccccgaaccggtgaccgtgtcctggaacagcggagccctgaccagcggcgtgcacaccttccccgccgtgctgcagagcagcggcctgtacagcctgagcagcgtggtgaccgtgcccagcagcagcctgggcacccagacctacatctgtaacgtgaaccacaagcccagcaacaccaaggtggacaagaaggtggagcccaagagctgtgacaagacccacacctgccccccctgccctgcccccgagctggccggagcccccagcgtgttcctgttcccccccaagcctaaggacaccctgatgatcagcagaacccccgaggtgacctgtgtggtggtggatgtgagccacgaggaccctgaggtgaagttcaactggtacgtggacggcgtggaggtgcacaatgccaagaccaagcccagggaggagcagtacaacagcacctaccgggtggtgtccgtgctgaccgtgctgcaccaggattggctgaacggcaaggagtacaagtgtaaggtgtccaacaaggccctgcctgcccctatcgagaaaaccatcagcaaggccaagggccagcccagagagccccaggtgtacaccctgccccctagcagagatgagctgaccaagaaccaggtgtccctgacctgcctggtgaagggcttctaccccagcgacatcgccgtggagtgggagagcaacggccagcccgagaacaactacaagaccaccccccctgtgctggacagcgatggcagcttcttcctgtacagcaagctgaccgtggacaagagcagatggcagcagggcaacgtgttcagctgctccgtgatgcacgaggccctgcacaatcactacacccagaagagcctgagcctgtcccctggcaag